(19) United States
(12) Patent Application Publication
(10) Pub. No.: US 2012/0289749 A1
(43) Pub. Date: Nov. 15, 2012

(54) Gold Nanocatalysts and Methods of Use Thereof

(76) Inventors: Tewodros Asefa, Somerset, NJ (US); Ankush V. Biradar, Pune (IN)

(21) Appl. No.: 13/467,492

(22) Filed: May 9, 2012

Related U.S. Application Data
(60) Provisional application No. 61/484,040, filed on May 9, 2011.

(51) Int. Cl. B01J 31/02; B01J 37/16; C07C 45/28; B82Y 40/00; B82Y 30/00

(52) U.S. Cl. 568/311; 502/167; 568/385; 977/773; 977/890

(57) ABSTRACT

Nanocatalysts and methods of synthesizing and using the same are provided.

This application claims priority under 35 U.S.C. §119(e) to U.S. Provisional Patent Application No. 61/484,040, filed on May 9, 2011. The foregoing application is incorporated by reference herein.

This invention was made with government support under Grant Nos: CHE-1004218 and DMR-0968937 awarded by the National Science Foundation (NSF). The government has certain rights in the invention.

### FIELD OF THE INVENTION

The present invention relates to the field of catalysts. Specifically, efficient and selective nanocatalysts, methods of synthesis, and methods of use thereof are disclosed.

### BACKGROUND OF THE INVENTION

Several publications and patent documents are cited throughout the specification in order to describe the state of the art to which this invention pertains. Each of these citations is incorporated herein by reference as though set forth in full.

The use of metal nanoparticles (MNPs) in catalysis has rapidly increased in recent years because of their efficient and intrinsic size-dependent catalytic properties as well as their ability to catalyze a range of chemical reactions (Nishihata et al. (2002) Nature 418:164-167; Astruc et al. (2005) Angew. Chem., Int. Ed., 44:7852-7872; Moreno-Manas et al. (2003) Acc. Chem. Res., 36:638-643; Li et al. (2002) Langmuir 18:4921-4925; Ranu et al. (2009) Pure Appl. Chem., 81:2337-2354; Barbaro et al. (2010) Dalton Trans., 39:8391-8402; Migowski et al. (2006) Chem. Eur. J., 1:32-39; Durand et al. (2008) Eur. J. Inorg. Chem., 23:3577-3586). For many MNPs to catalyze reactions or result in efficient catalysis, the reacting substrates must directly interact with the metal surfaces. This metal-substrate interaction would be greater if the MNPs were synthesized “naked”. Unfortunately, however, atoms of “naked” MNPs have a greater tendency to aggregate into a bulk material due to their high surface energies, which results in loss of, or decrease in, their intrinsic catalytic activity and selectivity over time (Moulijn, et al. (2001) Appl. Catal. A: Gen., 212:3-16; Xing et al. (2007) Chem. Mater., 19:4820-4826). In particular, Pd nanoparticles (PdNPs), which are well known for their catalytic activities, can easily aggregate to form Pd-black because of the very high surface energy of palladium (Iwasawa et al. (2004) J. Am. Chem. Soc., 126:6554-6555). Although the degree of aggregation of PdNP or other MNP catalysts can be overcome or minimized by passivating the metals' surfaces with organic ligands, this too will, unfortunately, be accompanied by the loss of catalytic activity because the very sites on the metals where catalysis takes place will be covered by these surface passivating organic groups (Jayamurugan et al. (2009) J. Mol. Catal. A: Chem., 307:142-148). Accordingly, there is a strong need for efficient and recyclable nanocatalysts.

### SUMMARY OF THE INVENTION

In accordance with the present invention, catalytically active nanoparticles are provided. In a particular embodiment, the nanoparticle comprises a mesoporous silica particle and Au nanoparticles. In a particular embodiment, the Au nanoparticles are contained within the mesopores of the silica particle, which are functionalized with a reducing agent (e.g., a hemiaminal or imine group). The nanoparticles may also comprise capping groups (e.g., methyl or alkyl groups) on the external surface of the silica particle.

In accordance with another aspect of the instant invention, methods of synthesizing the nanoparticle are provided. In a particular embodiment, the method comprises contacting mesoporous silica particles with oxidized Au (e.g., Au(III) or Au(I)), wherein the surface of the mesopores of the mesoporous silica particle are functionalized with a reducing agent. The method may further comprise synthesizing the mesoporous silica particles. In a particular embodiment, the mesoporous silica particles are synthesized by a) synthesizing silica particles in the presence of a surfactant, b) grafting the external surface of the silica particles with capping groups, c) removing the surfactant, and d) functionalizing the mesopores with a reducing agent.

In accordance with yet another aspect of the instant invention, methods of catalyzing a chemical reaction are provided. In a particular embodiment, the method comprises adding at least one nanoparticle of the instant invention to the chemical reaction. In a particular embodiment, the chemical reaction is an oxidation reaction, particularly one that leads to the ketone formation on an alkane (e.g., linear alkane or aryl substituted alkane). In a particular embodiment, the method comprises performing multiple rounds (e.g., 2 or more, 3 or more, etc.) of the chemical reaction with the same catalytic nanoparticles.

### BRIEF DESCRIPTIONS OF THE DRAWING

FIG. 1 provides transmission electron microscope (TEM) images of Au/SBA-15 catalysts (a) A, (b) B and (c) C that were prepared from 0.01, 0.1 and 1.0 mM, respectively, of aqueous HAuCl₄ solutions. Graphs of the average size of the Au nanoparticles are also provided.

FIG. 2 provides ¹³C CP-MAS NMR spectra of as-synthesized SBA-15 material whose external surface has been functionalized with methyl (-Me groups), before (Bottom) and after (Top) calcination at 350° C. The calcination step removed the P123 groups, leaving the Me groups, which are visible in the Top spectrum.

FIG. 3 provides transmission electron microscopy (TEM) images of Au/SBA-15 nanocatalysts that were prepared from SBA-15 material containing no Me groups on its external surface. 0.5, 1.0 and 2.0 mM of HAuCl₄ solutions were used to produce these materials labelled as (a) A′, (b) B′ and (c) C′, respectively.

FIG. 4 provides nitrogen gas adsorption/desorption isotherms of Au/SBA-15 nanocatalysts that were prepared from Me-SBA-15 material which contained Me groups on its external surface.

FIG. 5 provides nitrogen gas adsorption/desorption isotherms of Au/SBA-15 nanocatalysts that were prepared from SBA-15 material containing no Me groups on its external surface.

FIG. 6 provides thermogravimetric traces of Me-SBA-15, NH2-SBA-15, and Hemiaminal-SBA-15 samples.

FIG. 7 provides TEM image of Me-SBA-15 sample showing the highly ordered mesopores in it. The ordered mesoporous structures in the materials clearly remained intact after the postgrafting reaction.

FIG. 8 provides diffuse UV-Vis spectra of Au/SBA-15 samples A, B, and C showing the characteristic plasmon bands corresponding to Au nanoparticles in the range of ˜521 to ˜525 nm.

FIG. 9 provides powder X-ray diffraction (XRD) patterns of Au/SBA-15 samples A, B, and C.

FIG. 10 provides GC-MS spectra of reaction products for different catalytic reactions of alkane oxidation catalyzed by our Au/SBA-15 catalyst: a) ethylbenzene, b) diphenylmethane, c) propylbenzene, d) 1,3-diethylbenzene, and e) n-hexane.

FIG. 11A provides GC chromatogram of the reaction products from the oxidation reaction of n-hexadecane catalyzed by our Au/SBA-15 catalyst. FIG. 11B provides an expanded view of the GC chromatogram showing the 2-, 3- and 4-hexadecanone products.

FIG. 12 provides GC-MS spectra of the three ketones (A) 2-hexadecanone, (B) 3-hexadecanone, and (C) 4-hexadecanone produced from the oxidation reaction of n-hexadecane catalyzed by our Au/SBA-15 catalyst.

FIG. 13 provides a mechanism for Au/SBA-15 catalyzed oxidation of alkane (ethylbenzene) into a predominant ketone product with a minor alcohol products and some t-BuOH by-product.

### DETAILED DESCRIPTION OF THE INVENTION

Herein, the synthesis of nanoporous silica supported gold nanoparticle catalysts is reported along with their efficient catalytic activities in oxidation of various substituted alkylbenzenes and linear alkanes. The Au nanoparticles are synthesized by reducing Au(III) ions in situ within the nanopores of hemiaminal-functionalized mesoporous silica using the hemiaminal groups as reducing agents. The resulting mesoporous silica-supported gold nanoparticles efficiently catalyze the oxidation reactions of various alkyl-substituted benzenes and linear alkanes using t-butyl hydroperoxide (TBHP) as an oxidant. The catalytic reactions yield up to ˜99% reactant conversion and up to ˜100% selectivity to ketone products in some cases. This high selectivity to ketone products by the catalysts is unprecedented, especially considering the fact that it is achieved under mild reaction conditions and without using any additives in the reaction mixture. In the case of n-hexadecane oxidation, the catalytic reactions generate no alcohol byproducts, unlike other similar catalytic systems that are recently reported in the literature. Recyclability and leaching tests for the catalyst are also included. The possible reaction mechanism for this Au-nanoparticle catalyzed alkane oxidation with TBHP oxidant into a ketone product with some minor alcohol byproducts is proposed. The reactions leading to the products appear to take place through two major steps, involving several radical intermediates that lead to a ketone and an alcohol.

Oxidative catalysis is an important route for the synthesis of many commodity chemicals as well as perfumes, drugs and pharmaceuticals (Caron et al. (2006) Chem. Rev., 106:2943-2989). In particular the oxidation of alkanes, which are relatively more abundantly available, produces a number of more valuable commodity chemicals. However, alkane oxidation still remains to be one of the most difficult reactions to perform because it involves harsh reaction conditions, or it requires corrosive chemical reagents such as potassium permanganate, potassium dichromate or ammonium cerium nitrate (Punniyamurthy et al. (2005) Chem. Rev., 105:2329-2363; Sheldon et al., Green Chemistry and Catalysis. (Wiley-VCH Verlag GmbH & Co KgaA, Weinheim.) 2007; Clerici et al. (1998) Catal. Today, 41:351-364). Thus, currently one of the major efforts in oxidative catalysis research is finding or developing effective alkane oxidation catalysts that can efficiently catalyze the oxidation of alkanes through activation of their C—H bonds. The second major effort in oxidative catalysis is the design and development of catalysts that can operate under mild reaction conditions and generate selectively the desired product.

Among the various alkanes used as substrates in alkane oxidation reactions, linear and phenyl-substituted alkanes such as ethylbenzene stand out as among the most important ones because their oxidation products are essential precursors for many types of pharmaceuticals and synthetic materials. For instance, the oxidation products of ethylbenzene such as acetophenone and 1-phenylethanol are useful as precursors in the synthesis of optically active alcohols, benzalacetophenones (chalcones) and hydrazones (Mehler et al. (1994) Tetrahedron Asym., 5:185-188; Blickenstaff et al. (1995) Bioorg. Med. Chem., 3:917-922; Newkomeand et al. (1966) J. Org. Chem., 31:677-681).

Current industrial practice of ethylbenzene oxidation is performed via thermal autoxidation, in the absence of any catalyst, often to produce ethylbenzene hydroperoxide, among other things. Furthermore, to date very few catalysts have been explored for oxidation of ethylbenzene (Ma et al. (2007) Catal. Lett., 113:104-108; Lu et al. (2010) J. Mol. Catal. A: Chem., 331:106-111; Toribio et al. (2009) Appl. Catal. A: Gen., 363:32-39; Orlińska, B. (2010) Tetrahedron Lett., 51:4100-4102; Shilov et al. (1997) Chem. Rev., 97:2879-2932). Furthermore, these previously reported catalysts have relatively weak catalytic activities in ethylbenzene oxidation, or they require comparatively harsh reaction conditions. For instance, mesoporous silica functionalized with cobalt(II) oxide (Co/SBA-15) was reported to catalyze the oxidation of ethylbenzene; however, the catalytic reaction was shown to work only at relatively high temperatures (120 to 150° C.), giving only moderate % conversion of ethylbenzene—the highest reported value being 70.1% in 9 hours at 150° C. In addition, this catalyst was reported to form mixed uncontrolled oxidation products such as 1-phenylethyl hydroperoxide, benzoic acid, acetophenone and 1-phenylethanol (Ma et al. (2007) Catal. Lett., 113:104-108). In another example, the oxidation of ethylbenzene with hydrogen peroxide as an oxidant was shown to be catalyzed by the homogeneous catalyst 8-quinolinolato manganese(III) complex (Lu et al. (2010) J. Mol. Catal. A: Chem., 331:106-111). However, this catalytic reaction was also reported to give very small (26%) conversion of ethylbenzene, even after using ammonium acetate and acetic acid as additives in the reaction mixture. In another report, the oxidation of ethylbenzene into its hydroperoxide was achieved under soft reaction conditions in air in the presence of N-hydroxyimides such as N-hydroxysuccinimide, N-hydroxymaleinimide or N-hydroxynaphthalimide (Toribio et al. (2009) Appl. Catal. A: Gen., 363:32-39). Furthermore, the yield of the reaction to a specific product, that is, peroxyethyl benzene, was shown to improve by the addition of a minute amount of sodium hydroxide into the reaction mixture. However, the product selectivity of this catalytic reaction is still less efficient to be utilized for many industrial applications. Improvement of the selectivity of alkane oxidations can be improved by using different compounds as additives. For instance, in Cu(II), Co(II) or Mn(II) salt-catalyzed oxidation reactions of isopropylaromatic compounds to their corresponding alcohol or ketone products, including acetophenone, the use of N-hydroxyphthalimide as an additive was shown to improve the reaction's selectivity (Orlińska, B. (2010) Tetrahedron Lett., 51:4100-4102). However, the use of additives to improve the selectivity of oxidative reactions makes the catalytic system more costly.

Besides these aforementioned metal salts, a few others transition metals in homogeneous form were also reported to catalyze the oxidation of various hydrocarbons, including alkylbenzene (Shilov et al. (1997) Chem. Rev., 97:2879-2932). Interestingly, gold, both in the form of metal complexes as well as in the form of nanomaterials, has increasingly become attractive in recent years for use as a catalyst for a broad range of oxidative catalytic reactions. For example, both Au(I) and Au(III) complexes have been successfully used as homogeneous catalysts for alkane oxidations (Shulpin et al. (2001) Tetrahedron Lett., 42:7253-7256).

Furthermore, the first report two decades ago on the use of Au (noble metal) nanoparticles as a heterogeneous catalyst for gas phase oxidation reactions was a fascinating research development in the field of catalysis (Haruta et al. (1987) Chem. Lett., 16:405-408). Many other papers have since then also appeared on the successful use of Au nanoparticles for the catalysis of various oxidation reactions (Hughes et al. (2005) Nature, 437:1132-1135; Sinha et al. (2004) Angew. Chem. Int. Ed., 43:1546-1548; Carrettin et al. (2002) Chem. Commun., 696-697; Xu et al. (2005) Catal. Lett., 101:175-179; Biella et al. (2002) J. Catal., 206:242-247; Carrettin et al. (2003) Phys. Chem. Chem. Phys., 5:1329-1336; Sinha et al. (2004) Top. Catal., 29:95-102; Biella et al. (2003) Inorg. Chim. Acta, 349:253-257; Bawaked et al. (2009) Green Chem., 11:1037-1044; Wittstock et al. (2010) Science 327:319-322; Oliveira et al. (2010) Green Chem., 12:144-149; Kidwai et al. (2010) Appl. Catal A: Gen., 387:1-4; Hu et al. (2011) Chem. Commun., 47:1303-1305; Dapurkar et al. (2009) Catal Lett., 130:42-47; Corma et al. (2008) Chem. Soc. Rev., 37:2096-2126). For instance, Au nanoparticles supported on graphite, SiO₂, or TiO₂ materials by deposition-precipitation method were reported to catalyze the epoxidation reaction of alkenes (Bawaked et al. (2009) Green Chem., 11:1037-1044). This deposition-precipitation method involved a step-wise process of deposition of Au(III) ions onto graphite, SiO₂, or TiO₂ support materials, followed by the reduction of the Au(III) ions into Au(0) (Bawaked et al. (2009) Green Chem., 11:1037-1044). More recently, a nanoporous gold catalyst prepared by etching Ag away from an AuAg alloy was shown to efficiently catalyze the oxidation of methanol to methyl formate at low temperatures (Wittstock et al. (2010) Science 327:319-322). The efficient catalytic activity of this material was proposed to be the result of the effective dissociation of O₂ over the nanoporous gold surface. In another work, the immobilization of Au nanoparticles within magnetic materials was demonstrated to produce easily separable supported Au nanocatalysts for alcohol oxidation reactions (Oliveira et al. (2010) Green Chem., 12:144-149). In addition, Au nanoparticles were successfully used as catalysts for the oxidation of secondary alcohols to ketones (Kidwai et al. (2010) Appl. Catal A: Gen., 387:1-4). The synthesis of thin gold nanowires has been shown along with their use for oxidation of alkene, which is more reactive system for oxidation than alkane, at 1 atm O₂ (Hu et al. (2011) Chem. Commun., 47:1303-1305). In another work, Au/TiO₂ system was used for ethylbenzene oxidation into the corresponding ketone under 1 atm O₂ at 90° C.; however, the reaction gave only 22% conversion with 22.1% selectivity of ketone (Dapurkar et al. (2009) Catal Lett., 130:42-47).

Despite these aforethentioned reports on Au nanoparticle-based catalysts and catalysis, the oxidative catalysis of alkyl-substituted benzenes and the selective oxidation of n-alkanes to ketone products efficiently by nanosized Au particles have not been demonstrated before. Ketones are versatile functional groups in organic chemistry and key intermediates for a number of products (Blickenstaff et al. (1995) Bioorg. Med. Chem., 3:917-922; Newkomeand et al. (1966) J. Org. Chem., 31:677-681). Thus their synthesis selectively in high yield from oxidation of alkanes would be tremendously important in various chemical processes. Furthermore, most of the previous reports on oxidative catalysis by Au nanoparticles have focused on alkene and alcohol substrates, which are relatively easier to oxidize than alkanes. In addition, many of the previously reported Au nanocatalysts were shown to work either under extreme conditions or catalyze reactions into a mixture of products consisting of alcohols, acids, aldehydes and ketones (Corma et al. (2008) Chem. Soc. Rev., 37:2096-2126; Chen et al. (2009) J. Am. Chem. Soc., 131:914-915; Wu et al. (2010) Microporous Mesoporous Mater., 141:222-230).

Herein, the synthesis and efficient catalytic activity of mesoporous silica supported-nanosized Au particles for oxidation of alkanes is reported, both in the form of alkyl-substituted benzenes and n-alkanes, using an oxidant such as TBHP. The mesoporous silica supported Au nanoparticles were prepared by an in situ hemiaminal reduction method. The Au nanoparticles were shown to efficiently catalyze the oxidation of various phenyl-substituted alkanes including ethylbenzene, as well as linear alkanes such as n-hexane and n-hexadecane at lower temperature (70° C.), producing selectively carbonyl (ketone) products.

The instant invention provides nanoparticles that with unexpectedly superior properties. The nanoparticles provided herein are efficient catalysts, exhibit high selectivity, and are recyclable without the loss of catalytic activity. The mesoporous silica supported Au nanoparticles and methods of synthesis are described in more detail hereinbelow. In a particular embodiment, the mesoporous silica supported Au nanoparticles of the instant invention have a diameter of less than about 1000 nm, less than about 750 nm, or less than about 500 nm. Compositions comprising at least one mesoporous silica supported Au nanoparticles of the instant invention and at least one carrier are also encompassed by the instant invention.

The nanoparticles of the instant invention comprise mesoporous silica, corrugated/nanoporous core-shell silica (e.g., etched (e.g., by KOH) silica microspheres; see, e.g., silica constructs of U.S. patent application Ser. No. 13/396,052), and/or porous titania (e.g., mesoporous titania) particles encompassing Au nanoparticles. While the instant application generally refers to mesoporous silica, these other silica and titania particles may be used in place of the mesoporous silica. The term “mesoporous” indicates that the material contains pores with diameters between about 2 and about 50 nm. In a particular embodiment, the mesoporous silica particles have pores with diameters from about 2 to about 25 nm, about 5 to about 25 nm, about 2 to about 15 nm, or about 5 nm to about 10 or 12 nm. In a particular embodiment, the mesoporous silica particles are generally spherical. Types of mesoporous silica include, without limitation, MCM- (e.g., MCM-41, MCM-48), SBA- (e.g., SBA-15, SBA-1, SBA-16), MSU- (e.g., MSU-X, MSU-F), KSW- (e.g., KSW-2), FSM- (e.g., FSM-16), HMM- (e.g., HMM-33), and TUD (e.g., TUD-1). In a particular embodiment, the mesoporous silica is SBA-15. In a particular embodiment, the mesoporous material wall thickness is about 0.5 to about 10 nm, about 1 to about 7 nm, or about 1.5 to about 6 nm. With regard to the core-shell nanospheres, the shells may range from about 2 to about 60 nm in thickness, particularly about 4 to about 40 nm in thickness. The cores of the core-shell nanospheres may range from about 50 to about 600 nm, particularly about 100 to 450 nm in diameter.

In a particular embodiment, the mesoporous silica of the nanoparticles of the instant invention comprises capping groups on their external surface. The capping group may be an alkyl capping group. Examples of capping groups include, without limitation, methyl, n-propyl, n-pentyl, and n-octadecyl groups. The mesoporous silica of the nanoparticles of the instant invention may also comprise a reducing agent (e.g., a mild reducing agent) attached to the mesopore channel surface. In a particular embodiment, the reducing agent is a hemiaminal group (i.e., a functional group that comprises a hydroxyl group and an amine attached to the same carbon atom (C(OH)(NR₂), wherein R is H or alkyl). The reducing agent may also be an imine. In a particular embodiment, the imine is a functional group comprising the structure R₃—N═C(R₁)R₂, wherein R₁, R₂, and R₃ are independently H or alkyl. In a particularly embodiment, the imine comprises the structure—(CH₂)ₙ—N═CH—(CH₂)ₘ—CH₃. In a particular embodiment, n is about 1 to about 10 or about 1 to about 6 and m is about 0 to about 7 or about 0 to about 4.

The Au nanoparticles within the mesoporous silica particles may have a diameter small enough to fit within the mesopores. In a particular embodiment, the Au nanoparticles have a diameter from about 2 to about 25 nm, about 2 to about 15 nm, about 3 to about 10, or about 5 nm to about 10 nm. In a particular embodiment, the Au is Au(0) within the mesoporous silica particles.

The instant invention also encompasses methods of synthesizing the above nanoparticles. In a particular embodiment, the method comprises contacting mesoporous silica particles with a solution comprising oxidized Au (e.g., Au(III), Au(1), HAuCl₄), wherein the mesopore channel surface of the mesoporous silica particles is functionalized with a reducing agent (e.g., hemiaminal groups). The mesoporous silica particles may also comprise capping groups (e.g., organic capping groups such as methyl groups) on the external surface. The methods may also comprise synthesizing the mesoporous silica particles. In a particular embodiment, the method comprises a) synthesizing silica particles in the presence of a surfactant (e.g., poloxamers (i.e., block copolymers of (poly(ethylene oxide)-(poly(propylene oxide) or (poly(ethylene oxide)-(poly(propylene oxide)- (poly(ethylene oxide); e.g., Pluronic®-123; cetyltrimethylammoninum bromide; Brij 30; and the like), b) grafting the external surface of the silica particle with capping groups, c) removing the surfactant (e.g., via heat), and d) functionalizing the mesopore channels with a reducing agent (e.g., grafting the channels with amine and hydroxyl groups to yield hemiaminal functionalized silica).

The instant invention also encompasses methods of catalyzing a chemical reaction with the nanoparticles described herein. The nanoparticles may be used to catalyze, for example, oxidation reactions. In a particular embodiment, the nanoparticles are used to catalyze oxidation reactions of alkanes, including linear alkanes and aryl-substituted alkanes. The nanoparticles of the instant invention selectively catalyze the formation of ketones. In a particular embodiment, the reaction is performed in the presence of an oxidant, particularly TBHP. The reaction may be performed in any appropriate solvent, particularly a polar, aprotic solvent. Furthermore, based on the recyclable properties of the nanoparticles of the instant invention, methods comprising multiple rounds of chemical reactions without the need to replace or re-charge the catalyst are encompassed herein.

### DEFINITIONS

The following definitions are provided to facilitate an understanding of the present invention:

As used herein, the term “catalyst” refers to a substance that increases the rate of a chemical reaction while not being consumed in the reaction.

As used herein, the term “selective” refers to the capability of the catalyst to cause the production of specific products by selectively catalyzing a specific reaction, particularly in a mixture of similarly reactive compounds or from competitive reactions.

As used herein, the term “turnover number” refers to the number of moles of reactant that a mole of catalyst can convert to product before becoming inactivated.

A “carrier” refers to, for example, a diluent, adjuvant, preservative, anti-oxidant, solubilizer, emulsifier, buffer (e.g., Tris HCl, acetate, phosphate), water, aqueous solution, saline solution, dextrose and glycerol solutions, or vehicle with which a nanoparticle of the present invention can be contained.

The term “alkane” includes straight and branched chain hydrocarbons. Typically, an alkane will comprise 1 to about 20 carbons or 1 to about 10 carbons in the main chain. The hydrocarbon chain of the alkane may be interrupted with one or more oxygen, nitrogen, or sulfur. The alkane may, optionally, be substituted (e.g., with 1 to 4 substituents). Substituents include, without limitation, alkyl, alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol.

The term “aryl,” as employed herein, refers to monocyclic and bicyclic aromatic groups containing 6 to 10 carbons in the ring portion. Examples of aryl groups include, without limitation, phenyl, naphthyl, such as 1-naphthyl and 2-naphthyl, indolyl, and pyridyl, such as 3-pyridyl and 4-pyridyl. Aryl groups may be optionally substituted through available carbon atoms, preferably with 1 to about 4 groups. Exemplary substituents are described above. The aryl groups may be interrupted with one or more oxygen, nitrogen, or sulfur heteroatom ring members (e.g., a heteroaryl).

The following example provides illustrative methods of practicing the instant invention, and is not intended to limit the scope of the invention in any way.

### Example

### Experimental Procedures

### Materials and Reagents

Toluene, tetraethoxysilane (TEOS), 3-aminopropyltriethoxysilane (APTS, 99%), formaldehyde (37.2%), hexamethyldisilazane (HMDS), ethylbenzene, diphenylmethane, 1,3-diethylbenzene, propylbenzene, n-hexane, acetonitrile, methanol, tetrahydrofuran, ethyl acetate, t-butyl hydroperoxide (TBHP) and hydrogen peroxide (28%) were purchased from Sigma-Aldrich (St. Louis, Mo.), and they used as received without further purification. Anhydrous ethanol and hydrochloric acid were obtained from Fisher Scientific (Waltham, Mass.). HAuCl₄ was purchased from Strem Chemicals (Newburyport, Mass.). Pluronics®-123 ((PEO)₂₀(PPO)₇₀(PEO)₂₀) was obtained from BASF.

### Synthesis of SBA-15 and Me-SBA-15

SBA-15 was synthesized following the original procedure with a minor modification (Zhao et al. (1998) Science 279:548-552; Xie et al. (2008) J. Phys. Chem. C, 112:9996-10003). A solution of 12 g of Pluronic®-123 ((PEO)₂O(PPO)₇₀(PEO)₂₀, 313 g Millipore water and 72 g HCl (˜36 wt %) was prepared and stirred vigorously at 40° C. until all the Pluronic®-123 was dissolved. After adding 25.6 g of TEOS, the solution was stirred at 45° C. for 24 hours. The solution was then kept under static conditions at 80° C. in oven for another 24 hours to age. The resulting reaction mixture was filtered, and the precipitate was washed with copious amount of water and dried under ambient conditions. This produced as-synthesized SBA-15 mesostructured material. To graft the external surface of the mesostructured material with methyl groups, 4.0 g of this as-synthesized SBA-15 was suspended in a solution containing 30 mL of hexamethyldisilazane (HMDS) and 300 mL of toluene. The solution was then mildly stirred for 8 hours at room temperature in order to functionalize the external Si—OH groups of the as-synthesized SBA-15 with —Si(CH₃)₃ (or -Me) groups, and prevent possible growth of bigger metal nanoparticles on the outer surface of the mesoporous material from the reduction of Au(III) ions in the solution. The solid sample was recovered by filtration, washed with toluene and ethanol (2×10 mL in each case), and then let to dry under ambient conditions. This resulted SBA-15 sample with Me functional groups on its outer surface. It was then calcined in tube furnace at 350° C. for 5 hours under the flow of air to remove the Pluronics® template from its mesopores, without touching the Me groups (Xie et al. (2008) J. Phys. Chem. C, 112:9996-10003; Sun et al. (2006) J. Am. Chem. Soc., 128:15756-15764). The resulting mesoporous SBA-15, which was capped with external methyl groups and had free silanol groups in its mesopores, was denoted as Me-SBA-15.

### Synthesis of Hemiaminal-Functionalized SBA-15 (Hemiaminal-SBA-15)

The Me-SBA-15 synthesized above was dried in an oven for 12 hours at 80° C. before being grafted with amine groups. 1.5 g of the well-dried Me-SBA-15 was stirred in a solution of 4.5 mL of 3-amonopropyltriethoxysilane (APTS) in 120 mL of toluene for 6 hours at 80° C. to graft its mesoporous channel surface with primary amine groups. After filtration and washing with anhydrous ethanol (2×10 mL), the resulting sample (labelled as NH₂—SBA-15) was left to dry under ambient conditions. The NH₂—SBA-15 (1 g) material was then suspended in a mixture of 20 mL of ethanol and 10 mL of 37.2% formaldehyde solution at 40° C. for 1 hour. This produced a white colored, hemiaminal-functionalized mesoporous silica sample, denoted here as Hemiaminal-SBA-15.

In-Situ Synthesis of Au Nanoparticles within the Pores of Hemiaminal-SBA-15 (Au/SBA-5)

For the in-situ synthesis of Au nanoparticles within the mesoporous silica material, 50 mg of Hemiaminal-SBA-15 was dispersed in 10 mL of three different concentrations (0.01, 0.1 and 1.0 mM) of aqueous HAuCl₄ in a mixture of ethanol and water (1:4) and stirred for 30 minutes at 80° C. The resulting Au/SBA-15 samples, labelled as A, B, and C, respectively, were separated by filtration, washed with 20 mL water and then 10 mL ethanol, and let to dry under ambient conditions.

### Catalytic Oxidation Reaction

The catalytic oxidation reactions were carried out in a 50 mL three neck round bottom flask. In a typical oxidation reaction, 1 mmol alkane substrate, 15 mg Au/SBA-15 catalyst, 2 mmol (TBHP or H₂O₂) oxidant and 0.5 mL of chlorobenzene as an internal standard were mixed with a solvent (see Tables for different solvents used). The reaction was stirred with a magnetic stirrer. Samples were withdrawn after intervals of time and analyzed by gas chromatography (GC) and gas chromatography-mass spectrometry (GC-MS).

### Materials and Catalyst Characterizations

Nitrogen gas adsorption/desorption isotherms of all the mesoporous materials and catalysts were performed on Micromeritics TriStar® 3000 volumetric adsorption analyzer after degassing the samples at 160° C. for 12 hours. Thermogravimetric traces were collected on a TA Q50 Analyzer with a temperature ramping rate of 10° C./minute from room temperature to 780° C. under nitrogen gas flow. The UV-Vis absorption spectra of the Au/SBA-15 samples were measured with a Lambda 850 spectrophotometer (PerkinElmer; Waltham, Mass.). For the diffuse reflectance spectra measurement, the mesoporous powder samples containing the gold nanomaterials were sandwiched between two 3×3 cm quartz slides. Powder X-ray diffraction (XRD) patterns were recorded on a Siemens, Daco-Mp instrument having Cu—Kα radiation with wavelength of 1.54 Å. The diffractometer was set to 40 kV accelerating voltage and 30 mA. The XRD data were obtained by setting a wide scan range of 29 from 20° to 80° with step size of 0.015° and dwell time of 5 seconds. Transmission electron microscopy (TEM) images were obtained with a TOPCON microscope operated at 200 KV. The samples were prepared first by dispersing them in ethanol, casting a drop of the solution carbon/formvar coated Cu grids and letting them dry. The catalytic reactions were probed by withdrawing reaction mixtures in intervals of time and analyzing them by gas chromatography (GC) using an Agilent 6850 GC equipped with an HP-1 column (1% dimethyl polysiloxane, 30 m length, 0.25 mm internal diameter, 0.25 μm film thickness) and a flame ionization detector. The products were further confirmed by gas chromatography-mass spectrometry (GC-MS) (HP-5971) that was equipped with an HP-5 MS 50 m×0.200 mm×0.33 μm capillary column.

### GC Method

Detector: FID oven temperature: Starting temperature: 50° C. hold for 5 minutes then ramp 1: 30° C./minute up to 180° C. with hold time=1 minute, ramp 2: 30° C./minute up to 280° C. with hold time=5 minutes. Flow rate (carrier): 1.8 mL/minute (N₂) Split ratio: 50 Inlet temperature: 250° C. Detector temperature: 280° C.: GC was calibrated using chlorobenzene as an internal standard with R²: 0.9998 and conversion, selectivity and yield were calculated based on amount obtained.

Synthesis of Reference Au/SBA-15 Material from SBA-15, whose External Surface is not Passivated by Me Groups

SBA-15 material was prepared following the same procedure as reported (Zhao et al. (1998) Science 279:548-552; Xie et al. (2008) J. Phys. Chem. C, 112:9996-10003). A solution of 12 g of Pluronics®-123 ((PEO)₂₀(PPO)₇₀(PEO)₂₀, 313 g millipore water and 72 g HCl (˜36 wt %) was prepared and stirred vigorously at 40° C. until all the Pluronic®-123 was dissolved. Then, 25.6 g of TEOS was added into the solution and it was stirred at 45° C. for 24 hours. After this, the solution was kept under static conditions at 80° C. in oven for another 24 hours to age. The resulting reaction mixture was filtered, and the precipitate was washed with copious amount of water and dried under ambient conditions. This produced as-synthesized SBA-15 mesostructured material. The solid sample was recovered by filtration, washed with toluene and ethanol, and then let to dry under ambient conditions. This externally functionalized SBA-15 sample was calcined in tube furnace at 550° C. for 5 hours under the flow of air to remove the Pluronics® template, resulting in mesoporous SBA-15 with no organic capping groups on its external surface.

The SBA-15 sample was dried in an oven for 12 hours at 80° C. before being grafted with amine groups. Typically, 1.5 g of well-dried SBA-15 was stirred in a solution of 4.5 mL of 3-amonopropyltriethoxysilane (APTS) in 120 mL of toluene for 6 hours at 80° C. to graft its mesoporous channel surface with primary amine groups. After filtration and washing with anhydrous ethanol (2×10 mL), the resulting sample NH₂-functionalized SBA-15 was left to dry under ambient conditions and then let to react with 37.2% formaldehyde solution in anhydrous ethanol at 40° C. for 1 hour. This produced Hemiaminal-functionalized SBA-15.

This materials was then immobilized with Au(III) ions for the in-situ synthesis of Au nanoparticles within the SBA-15 mesoporous silica material. Typically, 50 mg of the Hemiaminal-functionalized SBA-15 was dispersed in 10 mL of three different concentrations (0.5, 1.0 and 2.0 mM) of aqueous HAuCl₄ in a mixture of ethanol and water (1:4) and stirred for 30 minutes at 80° C. The resulting Au/SBA-15 samples, labelled as A′, B′, and C′, respectively, were separated by filtration, washed with 20 mL water and then 10 mL ethanol, and let to dry under ambient conditions.

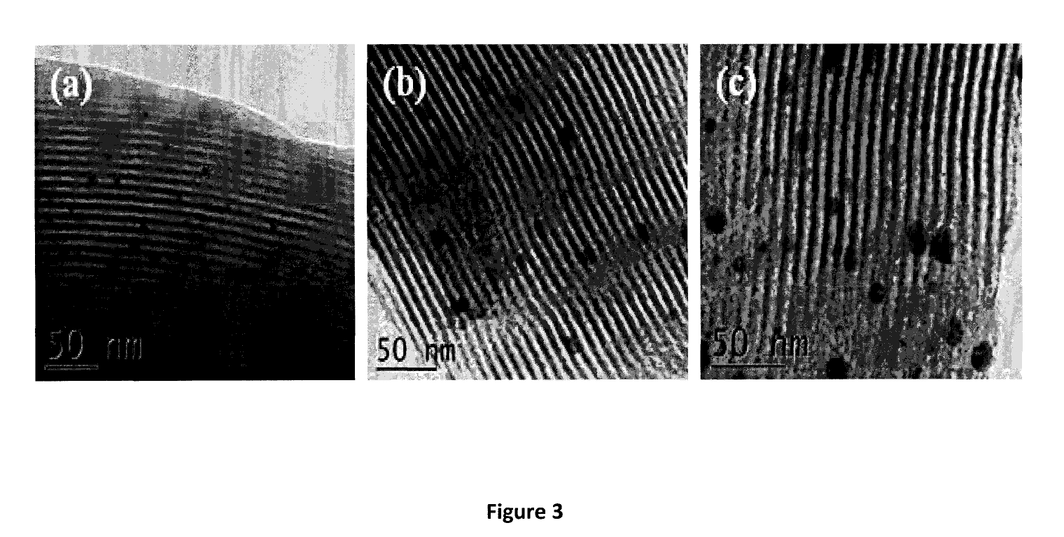

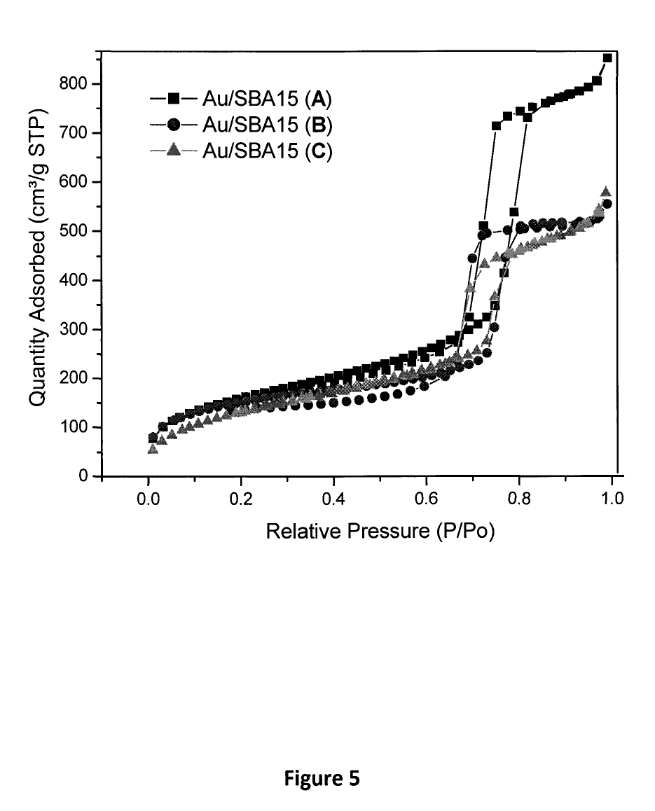

This attempted synthesis of the Au nanoparticles using an SBA-15 material that does not have Me groups on its external surface also resulted in Au nanoparticles; however, as can be seen in FIG. 3, the sizes of the Au nanoparticles at higher concentrations were bigger than the size of the channel of the mesoporous silica. This shows the importance of placing organic capping groups on the external surface of the SBA-15 to prevent possible growth of the Au nanoparticles on the outside surface. Their corresponding N₂ gas adsorption isotherms and pore size distributions are also shown in FIG. 5.

Since the sizes of most of the gold nanoparticles in sample C′ (FIG. 3C) are in the range of 8-14 nm and they appear to be bigger than the size of the mesopores of SBA-15 (˜9 nm). Thus, many of these particles should be outside the pores of the materials. Careful observation of the TEM images confirmed that this was the case. The Au nanoparticles may have been formed inside the pores of the mesoporous silica first, as in samples A′ and B′, but then diffused out as their sizes grew further because of the relatively larger concentration of HAuCl₄ used for the preparation of C′. Nonetheless, this sample, C′, with its bigger Au nanoparticles outside the mesopores allows for the investigation of the effect of size of Au nanoparticles in the oxidation reaction.

### Results

### Synthesis and Characterization

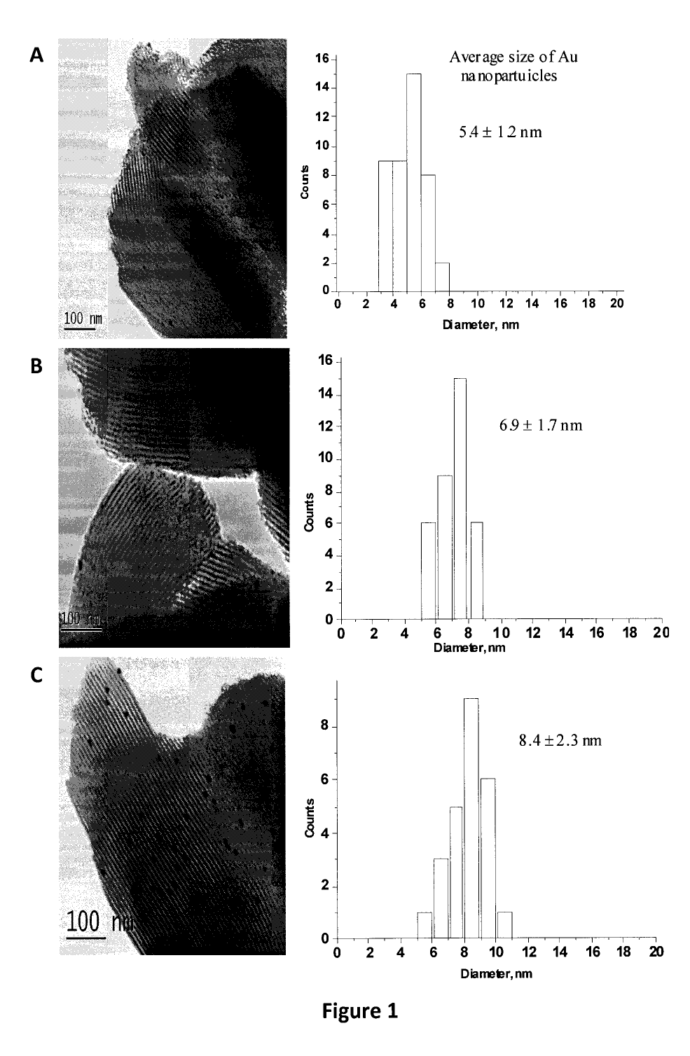

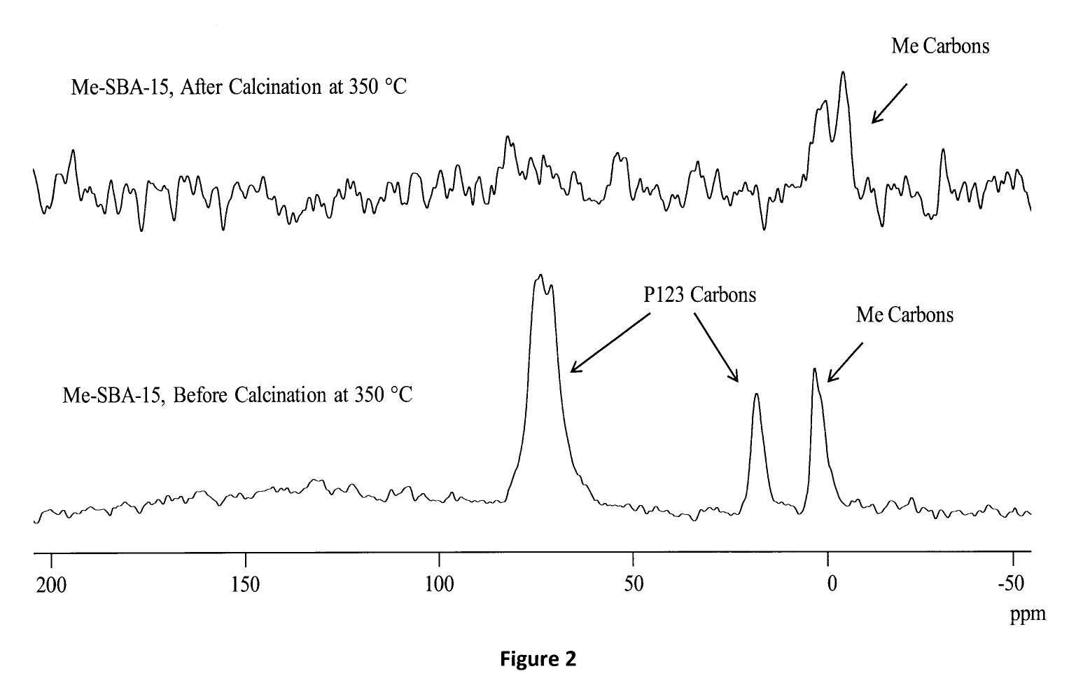

FIG. 1 displays transmission electron microscope (TEM) images of mesoporous silica-supported Au nanoparticles. The nanoparticles were synthesized by reducing Au(III) ions with hemiaminal groups that were tethered onto the mesopore channel surface of SBA-15 mesoporous silica (Xie et al. (2008) J. Phys. Chem. C, 112:9996-10003). To achieve this, first SBA-15 mesostructured silica was prepared (Zhao et al. (1998) Science 279:548-552; Xie et al. (2008) J. Phys. Chem. C, 112:9996-10003). Before surfactant extraction, the outer surface of the SBA-15 mesostructured silica was functionalized with methyl (-Me) groups using hexamethyldisilazane (HMDS). This produced mesostructured SBA-15 silica containing -Me groups on its outer surface. The removal of the surfactant templates at moderate temperature of 350° C. from the material resulted in reactive silanol (Si—OH) groups within its inner channel pores while leaving the -Me groups on the outer surface (FIG. 2) (Xie et al. (2008) J. Phys. Chem. C, 112:9996-10003; Sun et al. (2006) J. Am. Chem. Soc., 128:15756-15764). The temperature of 350° C. is chosen for calcination of the material because the Pluronics® templates undergo decomposition at this temperature, but not the -Me groups (Xie et al. (2008) J. Phys. Chem. C, 112:9996-10003; Sun et al. (2006) J. Am. Chem. Soc., 128:15756-15764). The resulting material was labelled as Me-SBA-15. This step allows for formation of small and more monodisperse Au nanoparticles, predominantly within the channel pores of the SBA-15. As shown in the TEM images in FIG. 3, without this step, bigger Au nanoparticles could form. After calcination, the hydroxyl groups formed in the mesopores of Me-SBA-15 were let to react with 3-aminopropyltriethoxysilane (APTS), and form 3-aminopropyl groups (or —NH₂ groups), only within the mesopores of the material. The —NH₂ groups in the resulting sample, denoted as NH₂—SBA-15, were then let to react with formaldehyde, and form surface grafted hemiaminal groups. The resulting sample was labelled as Hemiaminal-SBA-15.

Upon addition of Au(III) solution into Hemiaminal-SBA-15, large numbers of reasonably monodisperse Au nanoparticles within the channel pores of the material were formed from the reaction between the hemiaminal groups and the Au(III) ions. This resulted in the Au/SBA-15 samples (or Au/SBA-15 nanocatalysts). Three different concentrations, that is, 0.01, 0.1 and 1.0 mM, aqueous solutions of HAuCl₄ were stirred with 50 mg of Hemiaminal-SBA-15 for 30 minutes at 80° C. This produced three different Au/SBA-15 samples having different sized Au nanoparticles in them. The samples were labelled as A, B and C, respectively.

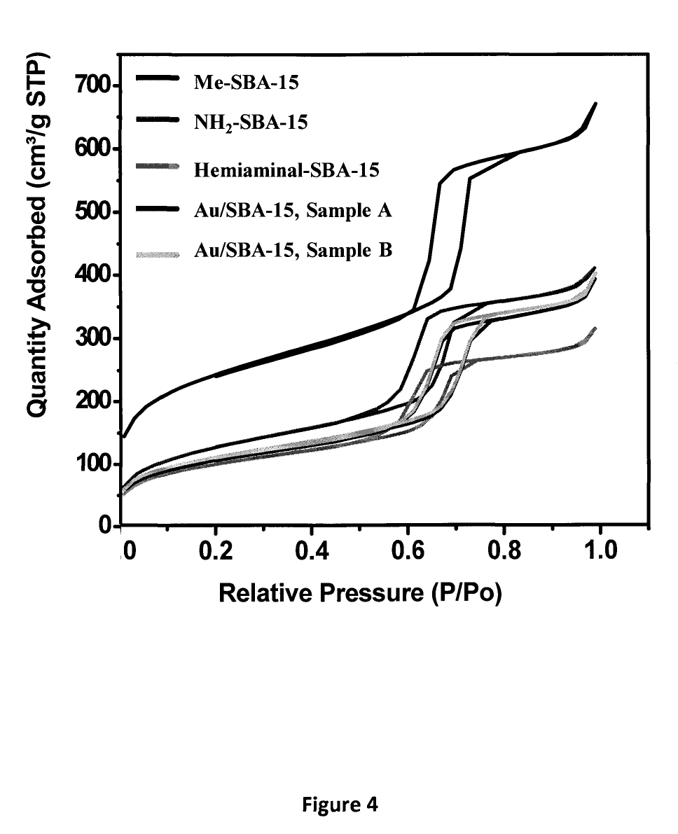

The Au/SBA-15 samples and their parent materials, including Me-SBA-15, NH₂—SBA-15 and Hemiaminal-SBA-15, were characterized by various spectroscopic and analytical methods. The N₂ gas adsorption measurements showed a type-IV isotherm for all the samples, indicating the presence of mesoporous structures in all the Au/SBA-15 samples as well as their parent materials (FIG. 4). For comparison purposes the N₂ gas adsorption isotherms of the reference Au/SBA-15 materials prepared from SBA-15, whose external surface is not capped with -Me groups, are also included in FIG. 5. The pore diameter of SBA-15 material before deposition of Au nanoparticles had monodisperse pore sizes with average Barret-Joyner-Halenda (BJH) pore diameter of ˜8.0 nm. Similarly, the pore size distributions of all the mesoporous materials after deposition of Au nanoparticles showed the presence of reasonably monodisperse mesopores; however, the average pore sizes decreased slightly to average BJH pore diameters with values ranging between 5.7 to 6.1 nm, presumably because the bigger pores had been filled with the deposited Au nanoparticles: The Brunauer-Emmett-Teller (BET) surface areas of Me-SBA-15, NH₂—SBA-15 and Hemiaminal-SBA-15 were 829, 449 and 348 m²g⁻¹, respectively. This indicates that there is a decrease in surface area as more organic groups are immobilized within the pores of the materials, as expected. The BET surface areas of the Au/SBA-15 samples A, B and C were 388, 381, and 373 m²g⁻¹, respectively.

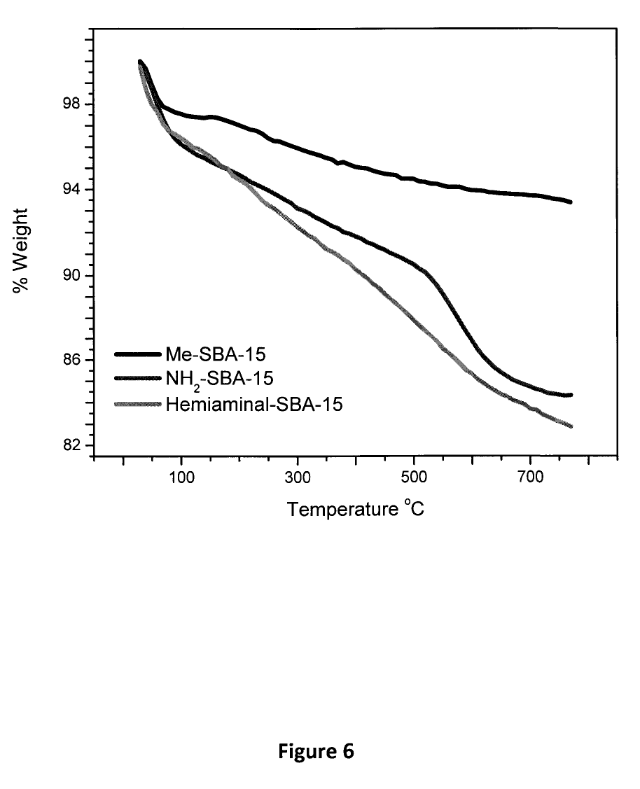

The thermogravimetric analysis (TGA) for the mesoporous samples Me-SBA-15, NH₂—SBA-15 and Hemiaminal-SBA-15 showed a weight loss below 100° C., which was attributed to the loss of physisorbed water (FIG. 6). In the temperature range of 100-550° C., the TGA traces showed weight losses of 3.4, 7.0, and 9.9% for samples Me-SBA-15, NH₂—SBA-15, and Hemiaminal-SBA-15, respectively. These weight reductions in the range of 100-550° C. were mainly due to the loss of methyl, organoamine, and/or hemiaminal groups from the samples upon heating. It can also be noted that the weight loss in the range of 100-550° C. from NH₂—SBA-15 was more than twice that from Me-SBA-15. This indicates the presence of more organic groups in the former due to the presence of both 3-aminopropyl and methyl groups in it. Similarly, the significantly higher weight loss from Hemiaminal-SBA-15 compared to that from NH₂—SBA-15 in the same temperature range was an indirect indication of the presence of the bulkier hemiaminal groups in place of the —NH₂ groups. The presence of all the different organic groups were further confirmed by elemental analyses and by ¹³C CP MAS NMR spectra shown in FIG. 2 (Xie et al. (2008) J. Phys. Chem. C, 112:9996-10003; Sun et al. (2006) J. Am. Chem. Soc., 128:15756-15764). Careful calculations based on the differences in weight losses seen in TGA for the different samples indicated that there were ˜0.97 mmol hemiaminal groups/g of Hemiaminal-SBA-15.

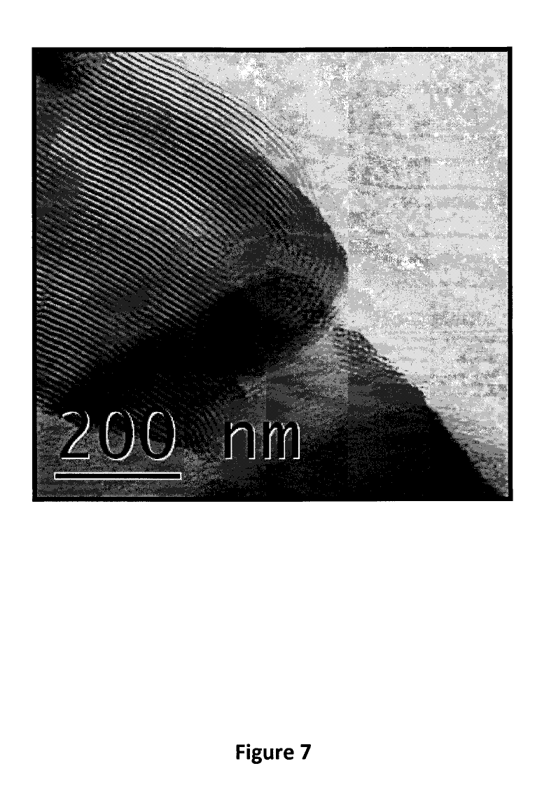

Further analysis by TEM (FIG. 1) showed the presence of reasonably monodisperse Au nanoparticles with average particle sizes of 5.4 (±1.2), 6.9 (±1.7) and 8.4 (±2.3) nm for Au/SBA-15 samples A, B and C, respectively. The formation of these different Au/SBA-15 samples has allowed us to investigate the effect of size of Au nanoparticles on their catalytic activities in alkane oxidation reactions (see below). The TEM images in FIG. 1 and the TEM images of the parent materials (for instance, of Me-SBA-15's that is shown in FIG. 7) also exhibit that the materials have well-ordered mesoporous channels.

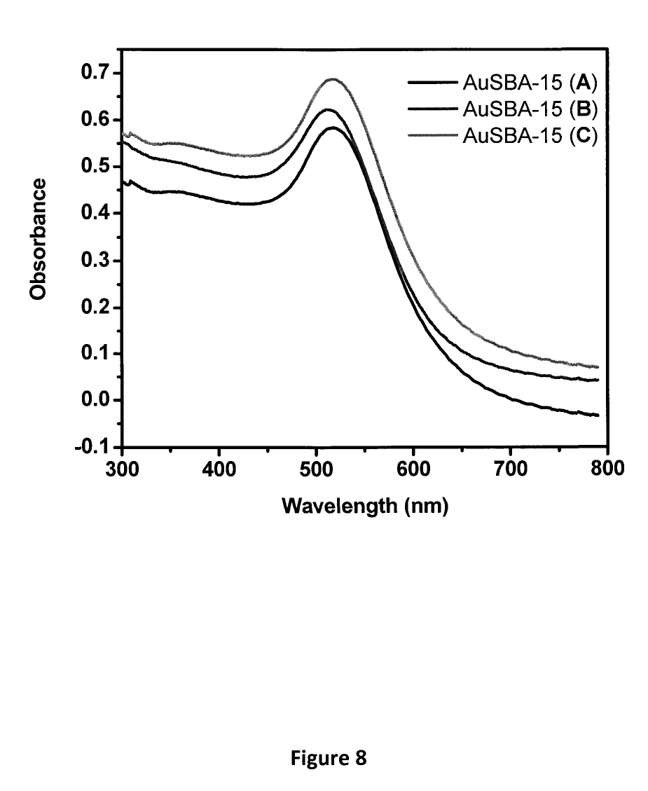

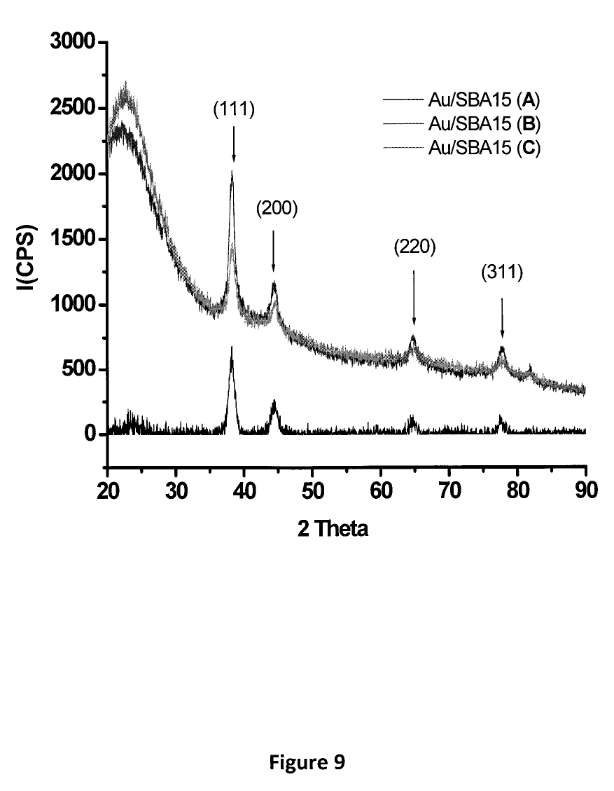

The formation of Au nanoparticles in the samples was further confirmed by diffuse UV-Vis spectroscopy by using the powdered Au/SBA-15 materials A, B, and C as samples (FIG. 8). The characteristic plasmon bands corresponding to Au nanoparticles were observed at ˜521 nm for samples A and B, but at ˜525 nm for sample C. The more blue-shifted absorption maxima for A and B compared to that of C indicates that the size of Au nanoparticles in samples A and B were slightly smaller than that in sample C, which is in agreement with the TEM results. In addition, powder X-ray diffraction (XRD) was used to characterize the Au nanoparticles of Au/SBA-15 samples (FIG. 9). The XRD patterns of all the Au/SBA-15 samples showed Bragg reflections at 2θ values of 38, 44, 64 and 77°. These Bragg reflections were indexed as the (111), (200), (220) and (311) diffracting planes, respectively, of metallic Au (Yan et al. (2005) Catal. Commun., 6:404-408). Careful inspection of the full-width-at-half-maxima (FWHM) of the Bragg reflections at 20 of 38° on the XRD patterns indicated that the peaks were slightly broader for A compared to B and C. This indicates that the Au nanoparticles in A were slightly smaller in size than those in B and C, which is consistent with the results obtained by TEM and UV-Vis analyses.

The presence of Au in the samples was further corroborated by ICP-AES which showed the presence of 1.08, 3.86 and 4.56 wt % Au in samples A, B and C, respectively. This corresponds to 54.8, 196.0, and 231.5 μmol Au/g of Au/SBA-15 (or 0.055, 0.196, and 0.232 mmol Au/g of Au/SBA-15). This clearly shows that the mol % of Au increases in the order of A<B<C, which is consistent with the amount of Au(III) used in the syntheses. However, the difference in wt % of Au produced between samples B and C was relatively smaller than that between A and B, although the corresponding difference in the mol of Au(III) used in the syntheses was the same. This is most likely due to the limited number of hemiaminals (or the reducing agents) present in the Hemiaminal-SBA-15 sample, causing a larger fraction of the Au(III) ions in case of C to remain unreduced. It is worth noting that 0.01, 0.10, and 1.00 mM concentrations of aqueous HAuCl₄ solution with 10 mL volume were used for 50 mg Hemiaminal-SBA-15 to synthesize Au/SBA-15 samples A, B and C, respectively. This implies that 0.1, 0.2, and 0.4 mmol Au(III), respectively, were used per gram of hemiaminal-SBA-15 sample. On the other hand, the hemiaminal-SBA-15 has a constant amount, 0.97 mmol, hemiaminals (or reducing agents) per gram of hemiaminal-SBA-15. Since three hemiaminals are required to reduce one Au(III) ion into Au(0), the 0.97 mmol hemiaminals/gram of hemiaminal-SBA-15 would be capable of reducing the theoretical maximum of only 0.32 mmol Au(III) into Au(0). This means, the amount of Au(III) used in case of C was slightly more than the available hemiaminal reducing agents in the hemiaminal-SBA-15 sample, which resulted in more incomplete reduction of the Au(III) used for during the synthesis of sample C. In fact, the filtrate from the synthesis of sample C was found to contain much more Au(III) than that in sample C or sample C by ICP-AES analysis.

### Catalytic Properties

The catalytic activities of the synthesized Au/SBA-15 materials A, B, and C were then tested in alkane oxidation reactions under similar conditions. Ethylbenzene, various other alkyl-substituted benzenes, n-hexane and n-hexadecane were used as model substrates. Ethylbenzene was chosen as a model substrate because its oxidation using Au nanoparticles as catalysts has never been reported previously. Furthermore, as mentioned above, the oxidation products from ethylbenzene are important precursors for a number of useful products (Mehler et al. (1994) Tetrahedron Asym., 5:185-188; Blickenstaff et al. (1995) Bioorg. Med. Chem., 3:917-922; Newkomeand et al. (1966) J. Org. Chem., 31:677-681). The other alkyl-substituted benzenes as well as n-hexane and n-hexadecane were used as substrates in order to demonstrate the versatility of the catalysts, investigate the scope of the catalytic reaction, and also evaluate the catalytic activity/selectivity of Au/SBA-15 with respect to other catalysts (Dapurkar et al. (2009) Catal Lett., 130:42-47).

As discussed above, the synthesis of Au/SBA-15 using different concentrations of HAuCl₄ produces samples containing different sized Au nanoparticles (or the samples labelled here as A, B and C). By using these three different Au/SBA-15 samples as catalysts, the effect of the size of the Au nanoparticles of Au/SBA-15 on their catalytic activities in the oxidation of alkanes, specifically alkylbenzene, was studied. Furthermore, the catalytic oxidation of ethylbenzene by Au/SBA-15 was investigated with the three commonly used oxidizing agents: air (O₂), H₂O₂ and TBHP (Table 1). Attempted oxidation of ethylbenzene with catalyst B in air, or with oxygen bubbled into the reaction mixture, as oxidant at 70 or 110° C. did not yield any oxidation product. Attempted oxidation of the reaction mixture, even at 300 Psig pressure of air in a Parr reactor, did not generate any oxidation product. When H₂O₂ was also used as an oxidant with the Au/SBA-15 catalyst, again no oxidation of ethylbenzene took place. The lack of oxidation with air or H₂O₂ was likely due to the use of larger Au nanoparticles. However, when TBHP was used as an oxidant, Au/SBA-15 was able to catalyze the oxidation of ethylbenzene efficiently. Furthermore, in a control experiment with SBA-15 and TBHP, ethylbenzene did not undergo any oxidation in 36 hours. Thus, the presence of Au/SBA-15 as catalyst and TBHP as oxidant allowed for ethylbenzene to undergo oxidation, indicating that the alkane oxidation reaction is catalyzed by the Au nanoparticles and with TBHP oxidant.

TABLE 1
Results for various oxidants on oxidation
of ethylbenzene by Au/SBA-15 (B).

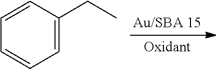

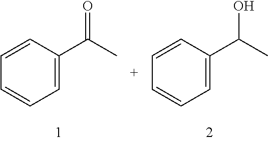

| | | % Conversion of | % Selectivity | | |
|---|---|---|---|---|---|
| Entry | Oxidant | Ethylbenzene | 1 | 2 | TON |
| 1 | 80% TBHP | 79 | 93 | 7 | 274 |
| | (aq.) | | | | |
| 2 | H₂O₂ | 0 | ~0 | ~0 | ~0 |
| 3 | Air (O₂) | 0 | ~0 | ~0 | ~0 |

Reaction condition: ethylbenzene: 1 mmol; oxidant: 2 mmol; solvent: acetonitrile: 10 mL; catalyst: (Au/SBA-15, sample B) and 15 mg overall mass; chlorobenzene (internal standerd): 0.5 mL; temperature: 70° C.; and reaction time: 36 hours. A schematic of the oxidation of ethylbenzene catalyzed by Au/SBA-15 is also provided.

It is worth noting, however, that TBHP itself can undergo some Au nanoparticle-catalyzed decomposition into t-BuOH. In fact, in the control experiment, where Au/SBA-15 catalyst B and TBHP are mixed, with no ethylbenzene, 11% of the TBHP was decomposed into t-BuOH. Thus, two equivalents of TBHP was used in the catalytic reactions in order to make up for any possible decomposition of TBHP and to ensure the presence of enough TBHP in the reaction mixture.

### Effect of Gold Nanoparticle Size on Catalytic Efficiency

Because TBHP was successfully served as an oxidant for ethylbenzene oxidation in the presence of our Au/SBA-15 catalyst, it was used in further studies below. For instance, in the presence of two equivalent of TBHP as oxidant, catalyst A, which contained 5.4±1.2 nm Au nanoparticles, resulted in 57% conversion of ethylbenzene, and gave a high selectivity (89%) to acetophenone product, with a minor (11%) 1-phenylethanol byproduct (Table 2 and FIG. 9). Au/SBA-15 catalysts B and C, which consisted of 6.9±1.7 and 8.4±2.3 Au nanoparticles, respectively, generated 79 and 89% conversions of ethylbenzene, with 93 and 94% selectivities, respectively, to acetophenone product. The remaining 6-7% byproduct was again the secondary alcohol 1-phenylethanol in both cases.

TABLE 2
Au/SBA-15-catalyzed oxidation of ethylbenzene. Three Au/SBA-15
catalysts A, B, and C having different size Au nanoparticles, which
were supported onto SBA-15, were used as catalysts. Reaction
condition: ethylbenzene: 1 mmol; 80% TBHP (aq.), 2 mmol; solvent:
acetonitrile, 10 mL; catalyst: Au/SBA-15 catalyst A, B or C with
15 mg overall mass; reaction temperature: 70° C.;
chlorobenzene (0.5 mL) used as internal standard (Hudlicy, T.
(2010) SYNLETT., 18: 2701-2707); reaction time: 36 hours; and
reaction atmosphere: air.

| | Catalyst or | Wt. % | | % Se- | | | |
|---|---|---|---|---|---|---|---|
| | Sample (Au | (mmol | % | lectivity | | | TOF |
| Entry | average size) | Au/g) | Conv. | 1 | 2 | TON | (h⁻¹) |
| 1 | SBA-15 | — | ~0 | ~0 | ~0 | ~0 | ~0 |
| 2 | Au/SBA-15 | 1.08% | 68 | 94 | 6 | 764 | 23 |
| | catalyst A | (54.8 μmol/g) | | | | | |
| | (5.4 ± 1.2 nm) | | | | | | |
| 3 | Au/SBA-15 | 3.86% | 79 | 93 | 7 | 274 | 8 |
| | catalyst B | (196.0 μmol/g) | | | | | |
| | (6.9 ± 1.7 nm) | | | | | | |
| 4 | Au/SBA-15 | 4.56% | 89 | 94 | 6 | 256 | 7 |
| | catalyst C | (231.5 μmol/g) | | | | | |
| | (8.4 ± 2.3 nm) | | | | | | |

Based on these results, one can conclude that catalyst C has a better catalytic efficiency and selectivity to a ketone product than catalysts A and B; and catalyst B, in turn, has better catalytic efficiency than catalyst A. However, when comparing the results based catalytic turn-over-numbers (TONs) and turn-over-frequencies (TOFs) (Table 1), an opposite trend in catalytic efficiencies was observed. That is, catalyst A gives significantly higher TON and TOF (764 and 23 h⁻¹) than catalyst B (274 and 8 h⁻¹); and sample B, in turn, gives higher TON and TOF than catalyst C (256 and 7 h⁻¹). This indicates that among the three different Au/SBA-15 catalysts studied for oxidation of ethylbenzene, the catalytic activity of A was actually greater than that of B or C when the catalytic activities were compared on the basis of catalytic activity per mol of Au. Because not all the supported Au nanoparticles such as those in the middle of mesopores, and because not all the atoms of the Au nanoparticles such as those in middle of the nanoparticles are exposed to participate in the catalytic reactions, the reported catalytic TONs and TOFs per total mol of Au are underestimations of the TON and TOFs.

The results in Table 2 also clearly indicate that the catalytic activities of Au nanoparticles of Au/SBA-15 catalysts in ethylbenzene oxidation vary with the size of the nanoparticles. This is also consistent with results in previous reports for other reactions involving oxidation of various organic substrates is shown to depend on the size of Au nanoparticles (Chen et al. (2009) J. Am. Chem. Soc., 131:914-915; Haider et al. (2008) Catal. Lett., 125:169-176; Chen et al. (2004) Science 306:252-255; Chen et al. (2006) Catal. Today, 111:22-33). For instance, the rate as well the selectivity of the Au nanoparticles in alcohol oxidation reactions are shown to be affected by the size of Au nanoparticles, with 6.9 nm-sized Au particles yielding the highest catalytic efficiency (Hudlicy, T. (2010) SYNLETT., 18:2701-2707). On the other hand, a smaller size (3.5-4.0 nm) Au nanoparticles are found to be the most effective in gas phase oxidation reactions, particularly CO oxidation (Chen et al. (2004) Science 306:252-255; Chen et al. (2006) Catal. Today, 111:22-33).

As shown in Table 3, the type of solvent used in ethylbenzene oxidation in the presence of Au/SBA-15 catalyst affects both the catalytic efficiency as well as the catalytic selectivity of the reaction. This was tested using different solvents with catalyst B at 70° C. for 36 hours. In the case of acetonitrile, 79% conversion of ethylbenzene with 93% selectivity to acetophenone product was obtained (Table 3, entry 1). When tetrahydrofuran (THF) was used as the solvent, a lower ethylbenzene conversion of 70% and a lower selectivity of 87% to acetophenone product were obtained (Table 3, entry 2). Upon using ethylacetate as the solvent, further decrease in the catalytic activity as well as selectivity to acetophenone resulted (Table 3, entry 3). In the case of toluene as the solvent (Table 3, entry 4), even lower catalytic activity and lower selectivity were obtained. This decrease in the catalytic activity of Au/SBA-15 in oxidation reaction in the order of acetonitrile>THF>ethylacetate>toluene might be the result of the lower degree of solubility of the reaction intermediates during the oxidation reactions in solvents with decreasing polarity or dielectric constant (Andrade et al. (2005) Current Org. Chem., 9:195-218). Generally, the dipolar, aprotic solvents such as THF gave better results than the non-polar solvents such as toluene. Most importantly, the reason that certain solvents work better than others seems to suggest that some of the solvents may undergo co-oxidation and produce a more oxidizing agent in the reaction. It has been previously reported that solvents such as methylcyclohexene work much better than any other solvent to form a peroxyl radical, which is then epoxidizing the substrate stylbene. Acetonitrile is known to produce peroxycarboximidic acid—a powerful oxidation agent. THF is also very well known to undergo radical oxidation. Hence, the ‘good’ solvents are probably speeding up the formation of radicals, or more importantly speeding up the chain length of the radicals, making them more available for the oxidation of the substrate ethylbenzene.

TABLE 3
Oxidation of ethylbenzene catalyzed by Au/SBA-15 (B) in different
solvents. Reaction condition: ethylbenzene: 1 mmol; 80% TBHP
(aq.): 2 mmol; solvent: different solvents as shown in the table,
10 mL; catalyst (Au/SBA-15, B), 15 mg; chlorobenzene (internal
standard): 0.5 mL; reaction temperature: 70° C.; reaction time:
36 hours; and reaction done in air.

| | |
|---|---|
| | % |
| % | Selectivity |

| Entry | Solvent | Conversion | 1 | 2 |
|---|---|---|---|---|
| 1 | Acetonitrile | 82 | 92 | 8 |
| 2 | Tetrahydrofuran | 70 | 87 | 13 |
| 3 | Ethyl acetate | 64 | 85 | 15 |
| 4 | Toluene | 45 | 84 | 16 |
| 5 | Methanol | 70 | 83 | 17 |

Since among all the solvents tested, acetonitrile resulted in the highest % conversion of ethylbenzene while giving the highest selectivity toward a particular product—in this case, a ketone (or acetophenone, 1)—all the other reactions for the subsequent studies (discussed below) were performed in acetonitrile.

TABLE 4
Oxidation of various alkyl-substituted benzenes
catalyzed by Au/SBA-15 catalyst, B.

| | | % | % Selectivity | |
|---|---|---|---|---|
| Entry | Reactant | Conversion | Ketone | Alcohol |
| 1 | 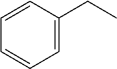 | 79 | 93 | 7 |
| 2 | 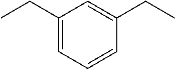 | 76 | 88 | 12 |
| 3 | 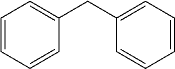 | 99 | 100 | 0 |
| 4 | 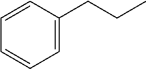 | 75 | 95 | 5 |
| 5 | 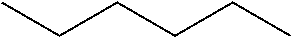 | 95 ^{b} | 92 | 8 |

Reaction conditions: substrate: 1 mmol; oxidant: TBHP, 2 mmol; catalyst (Au/SBA-15, B), 15 mg; solvent: acetonitrile, 10 mL; chlorobenzene (internal standard): 0.5 mL; reaction time: 36 hours; temperature: 70° C.
^{b} Similar condition as in (^{a}) except the product was collected a reaction time of 8 hours.

TABLE 5
Oxidation of n-hexadecane catalyzed by Au/SBA-15 (B) under various
reaction conditions. This reaction is particularly chosen to show both
the catalyst's versatility as well as relative efficiency and selectivity
compared to other closely related materials in similar reactions.

| | | | % Selectivity | | |
|---|---|---|---|---|---|
| | Reaction | | 2- | 4- | 3- |
| | Solvent or | % | hexa- | hexa- | hexa- |
| Entry | Condition | Conv. | decanone | decanone | decanone |
| 1 | In Acetonitrile^{b} | 9 | 58 | 41 | 1 |
| 2 | In Methanol^{b} | 5 | 57 | 42 | 1 |
| 3 | Neat^{c} | 15 | 40 | 47 | 13 |
| 4 | Neat^{d} | 74 | 42 | 47 | 11 |

^{b}Reaction condition: n-hexadecane (1 mmol) in 10 mL solvent (acetonitrile or MeOH); 80% TBHP (aq.), 2 mmol; catalyst: Au/SBA-15, B (15 mg); reaction temperature: 70° C.; chlorobenzene (internal standard): 0.5 mL and reaction time: 36 hours.
^{c}Reaction condition: n-hexadecane (25 mmol), neat and no solvent; 80% TBHP (aq.), 2 mmol; catalyst: Au/SBA-15, B (15 mg); reaction temperature: 70° C.; and reaction time: 24 hours.
^{d}reaction condition: n-hexadecane 25 mmol), neat and no solvent; 80% TBHP (aq.), 50 mmol; catalyst: Au/SBA-15, B, (15 mg); reaction temperature: 150° C. in a Parr reactor; and reaction time: 6 hours.

For instance, Au/SBA-15 catalyst B catalyzed the oxidation of 1,3-diethylbenzene with 80% conversion and 88% selectivity to 3-ethylacetophenone product in 36 hours. Interestingly, the catalytic oxidation of 1,3-diethylbenzene with Au/SBA-15 stopped after the oxidation of only one of its ethyl groups, and with no formation of 1,3-diacetophenone product in 36 hours (Table 4, entry 2). Au/SBA-15 sample B also catalyzed the oxidation of diphenylmethane with 99% conversion, and a remarkably high selectivity of ˜100% to benzophenone product (Table 4, entry 3). Furthermore, Au/SBA-15 catalyzed the oxidation of propylbenzene with 75% conversion and 95% selectivity to propiophenone, with 5% 1-phenyl-2-propanol byproduct (Table 4, entry 4).

When n-hexane was used as a substrate, catalyst B oxidized it with 95% conversion in 8 hours, giving 92% 2-hexanone as a major product (Table 4, entry 5). However, when this reaction was further continued for 30 hours, all the 2-hexanone was further converted into 2,4-di-hexanone product, and without resulting any alcohol or acid byproducts.

Catalytic Properties on n-alkanes

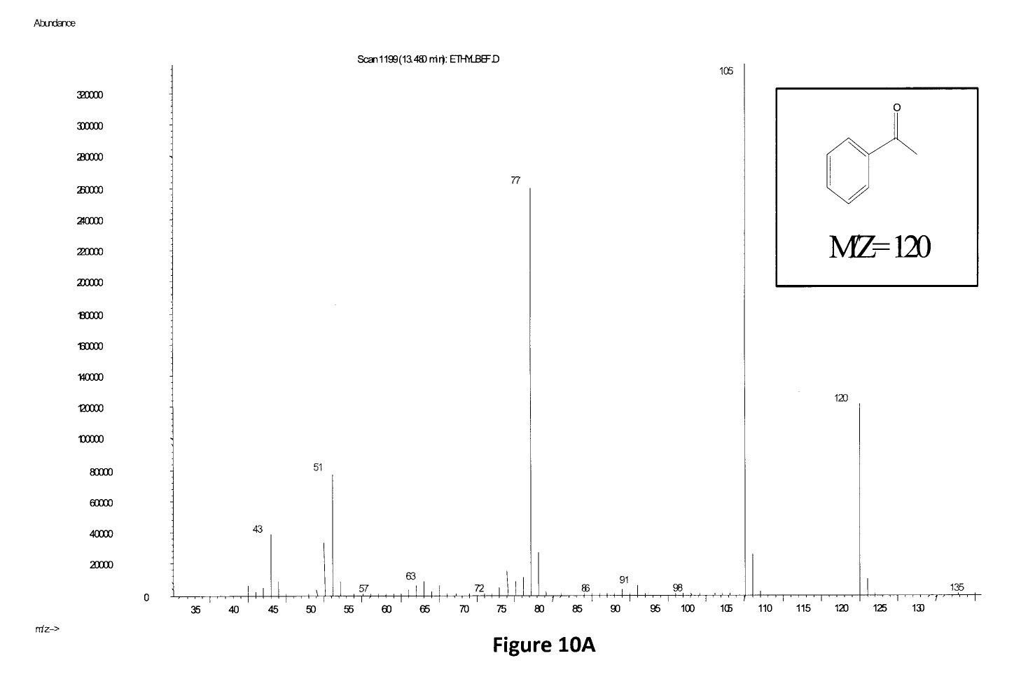

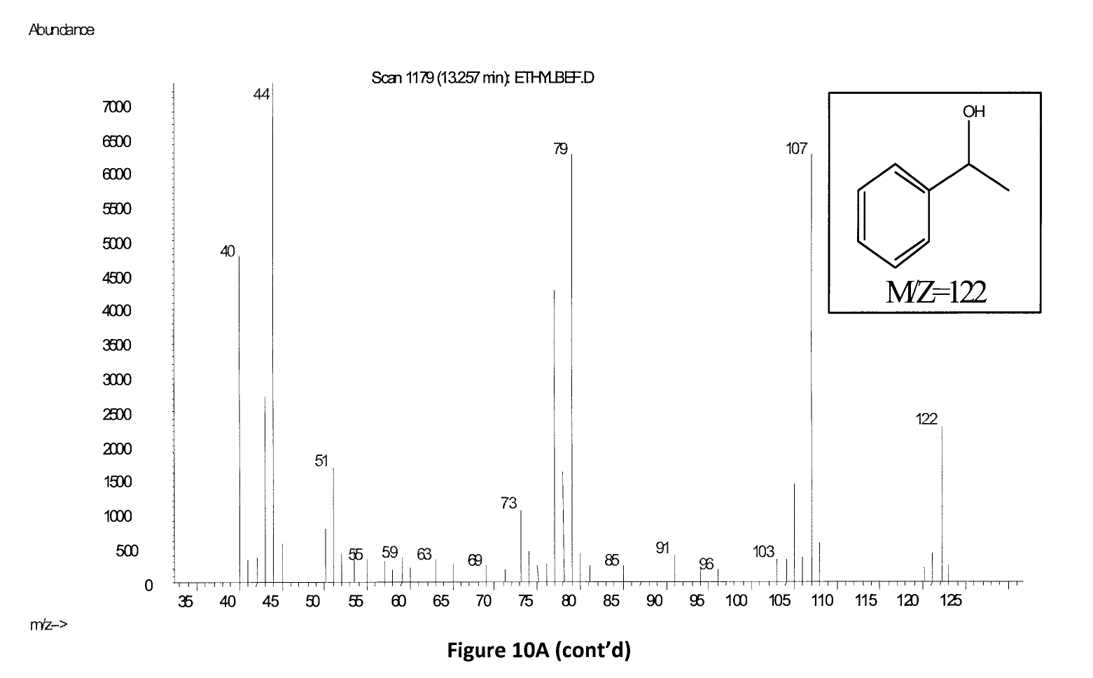

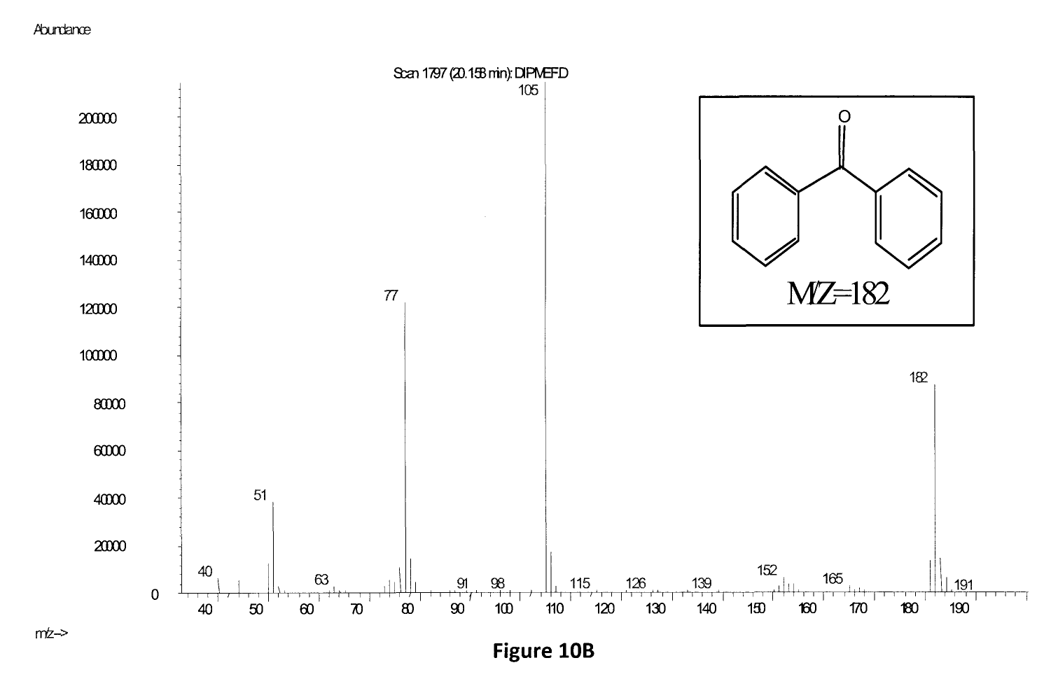

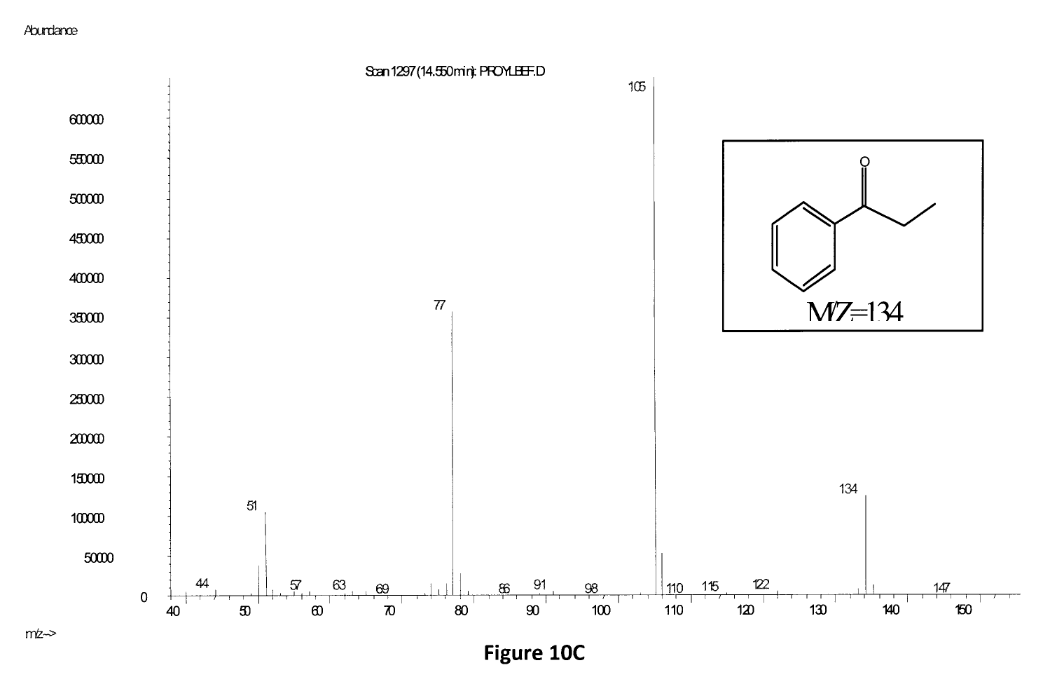

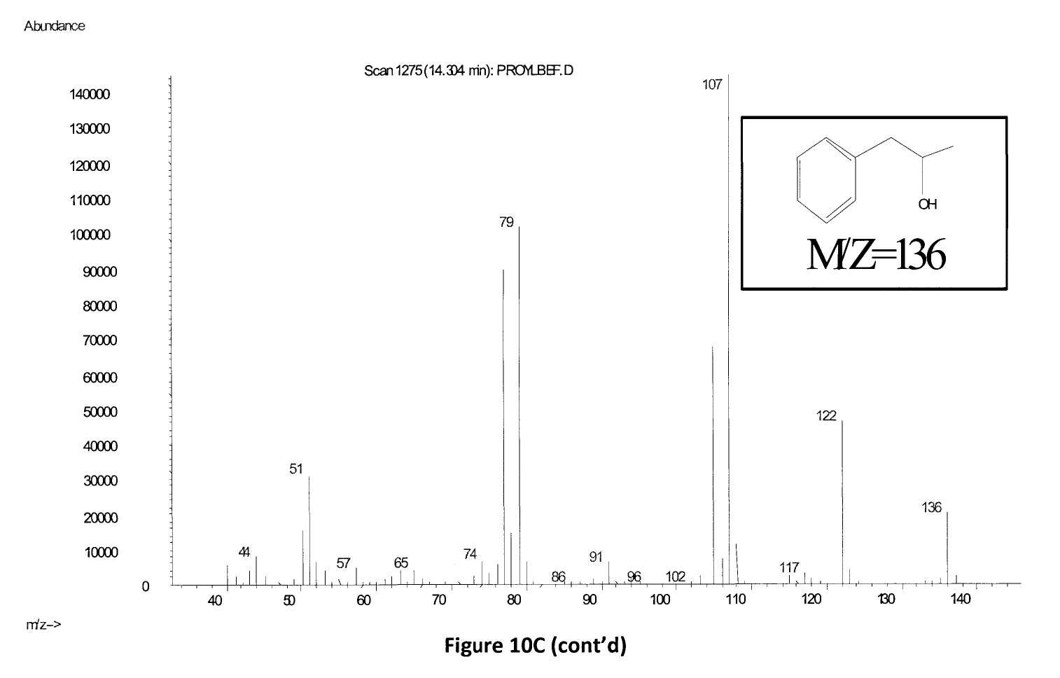

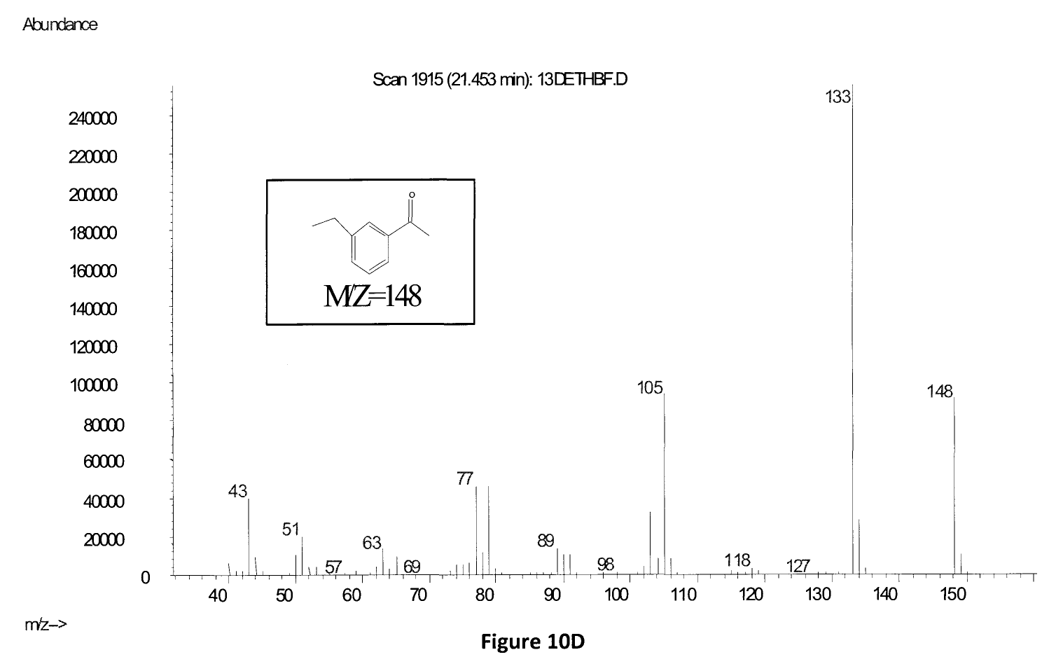

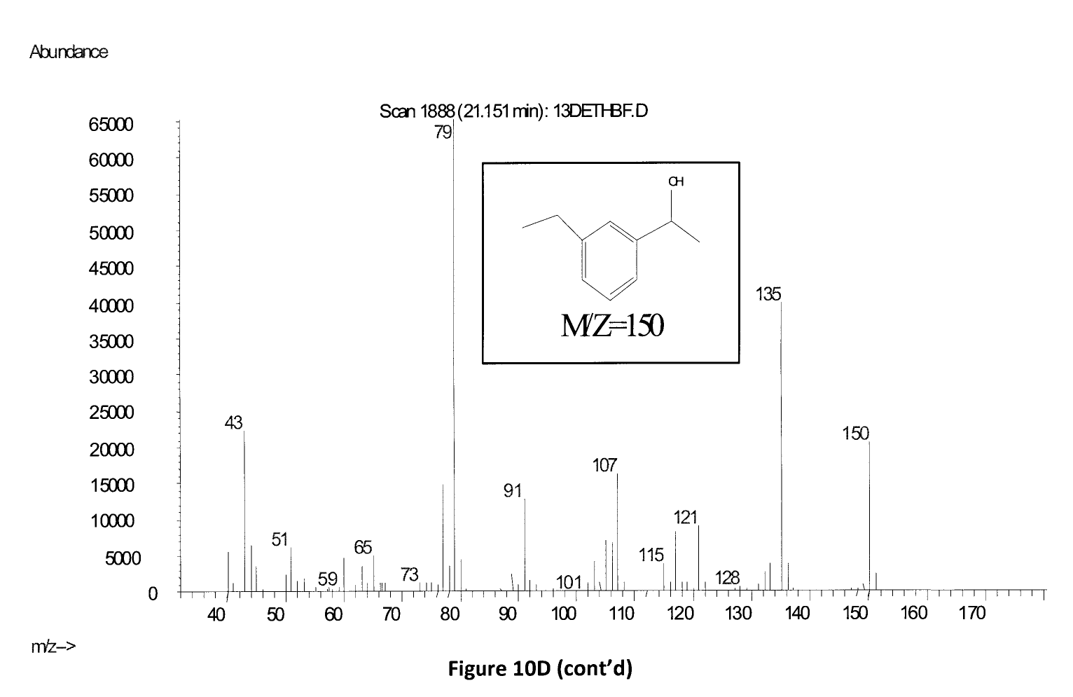

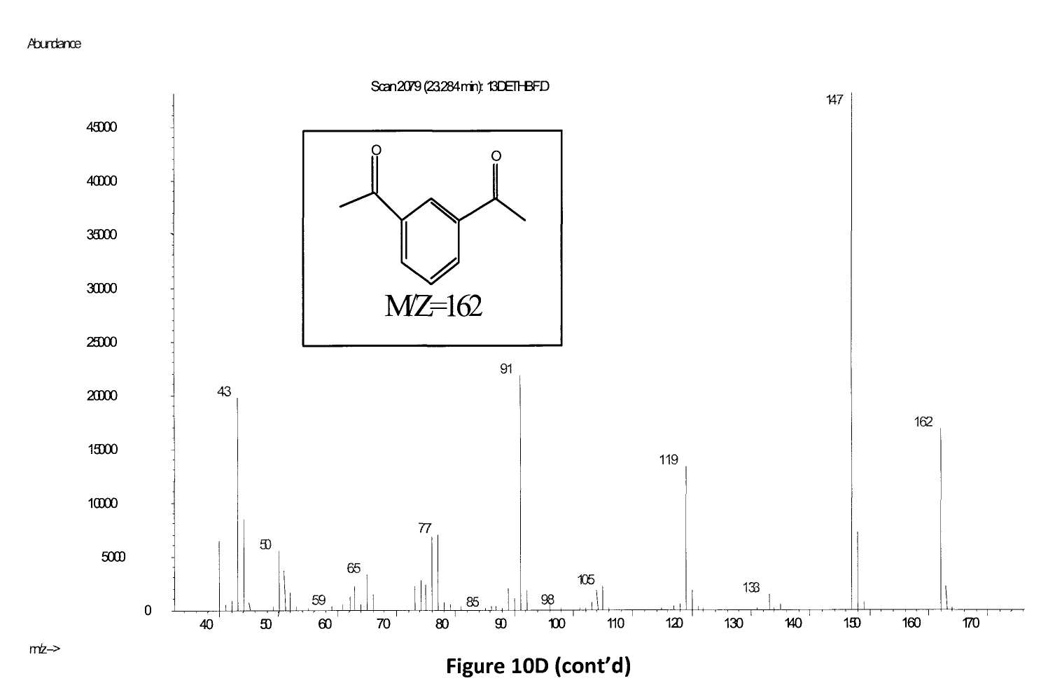

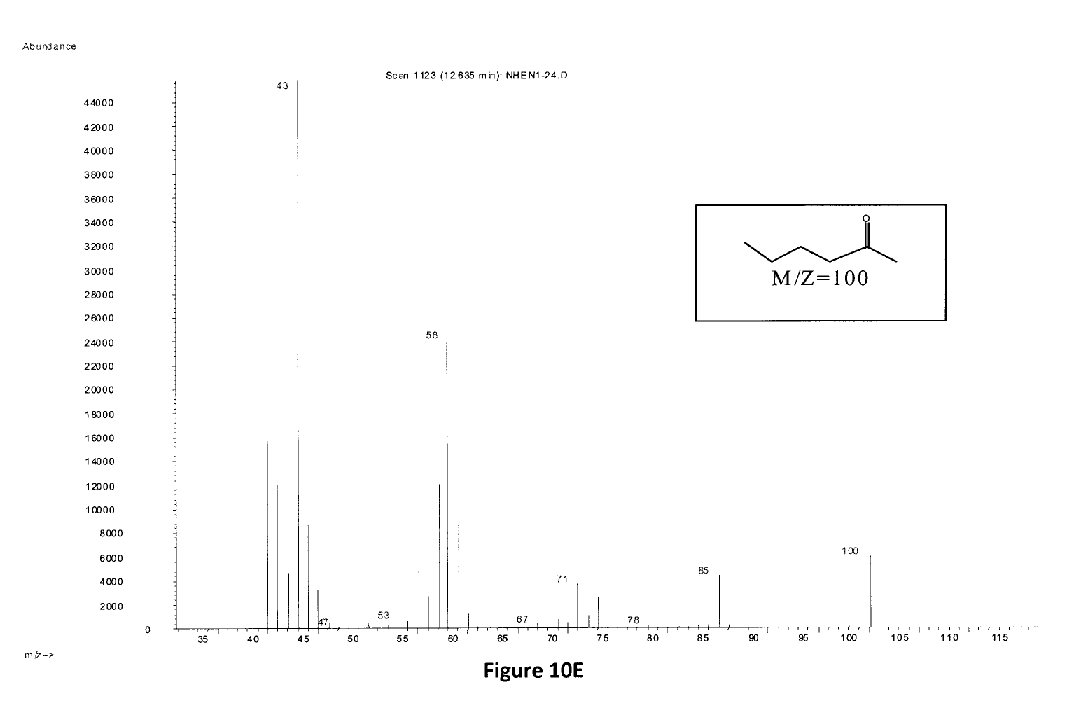

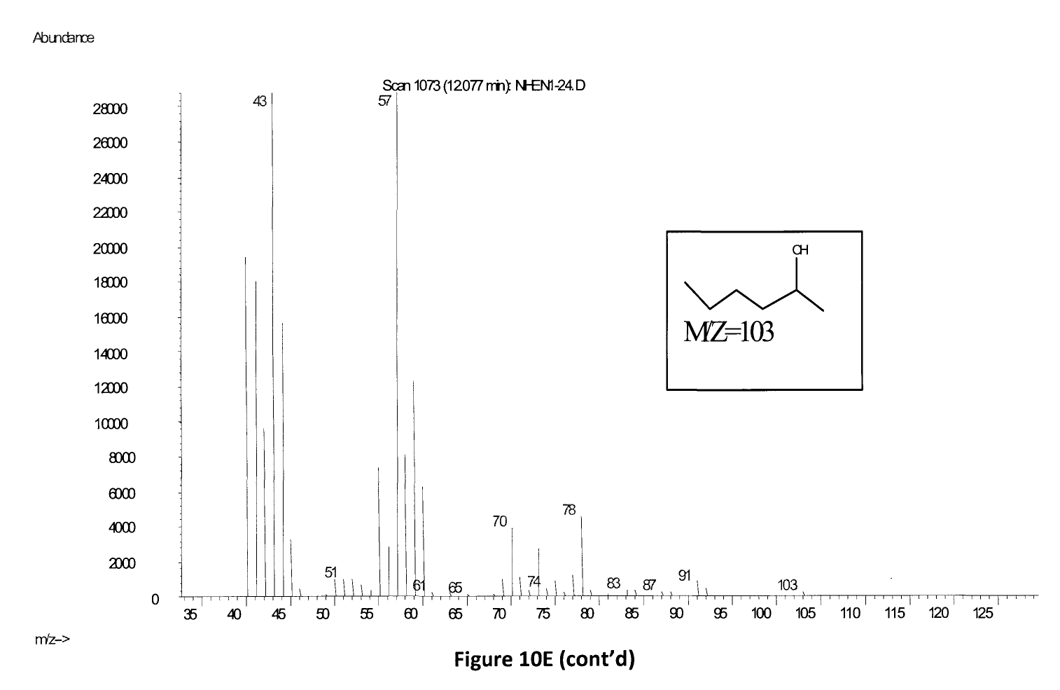

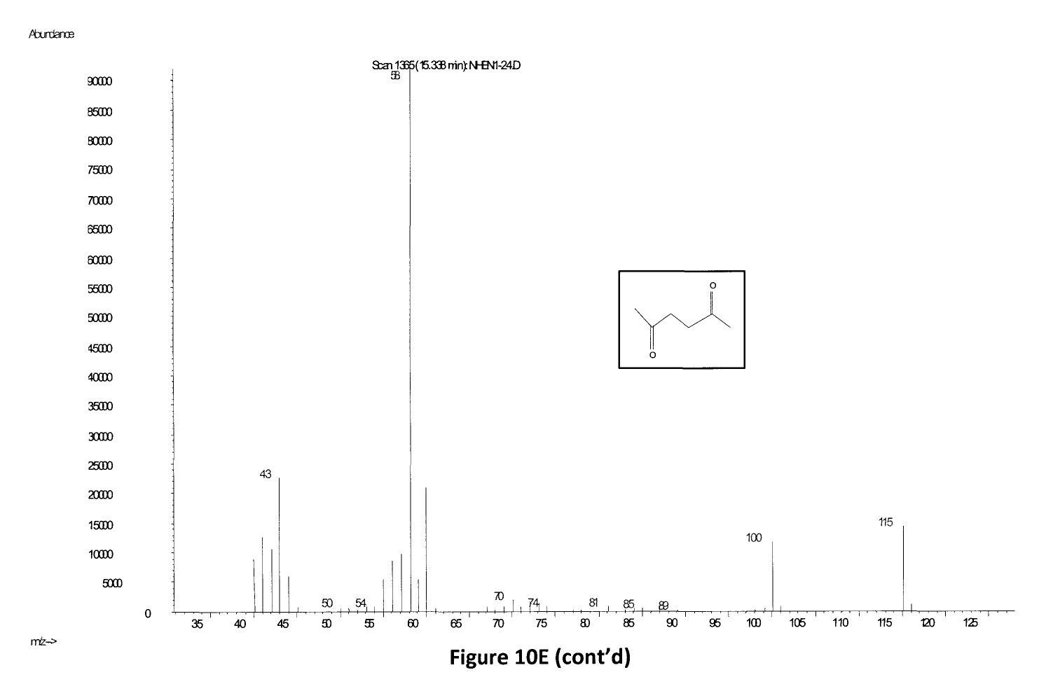

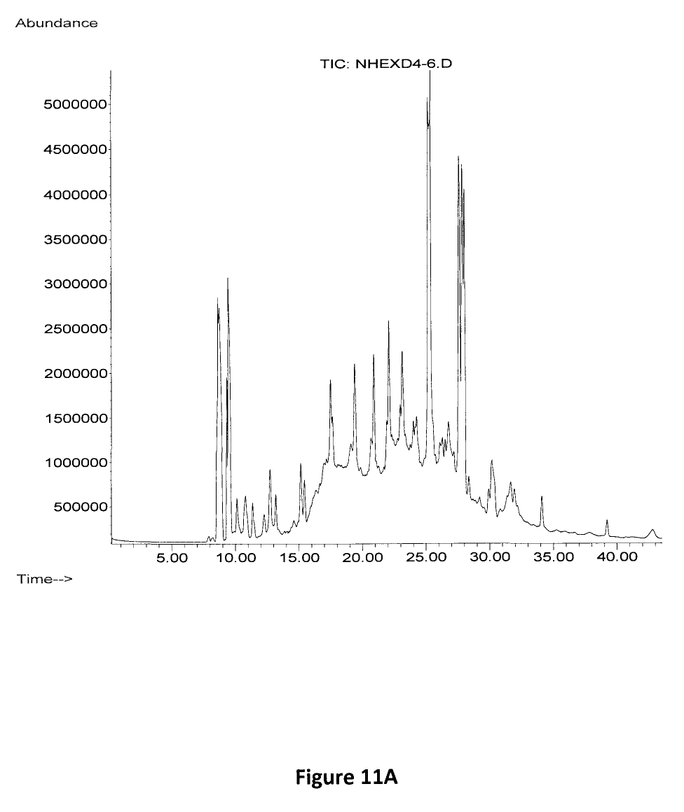

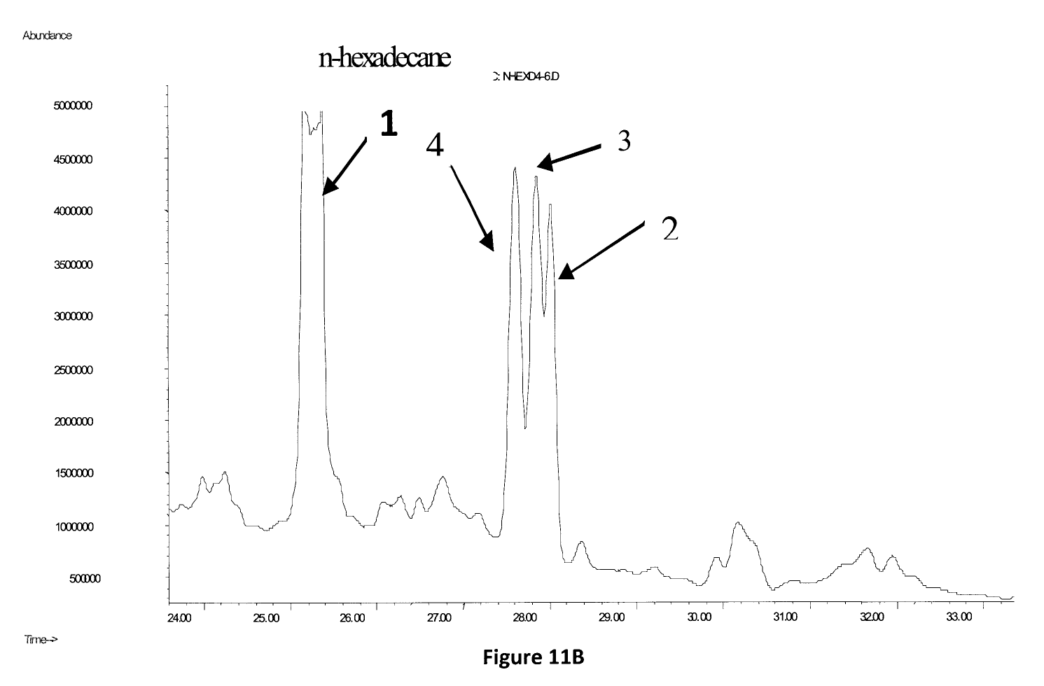

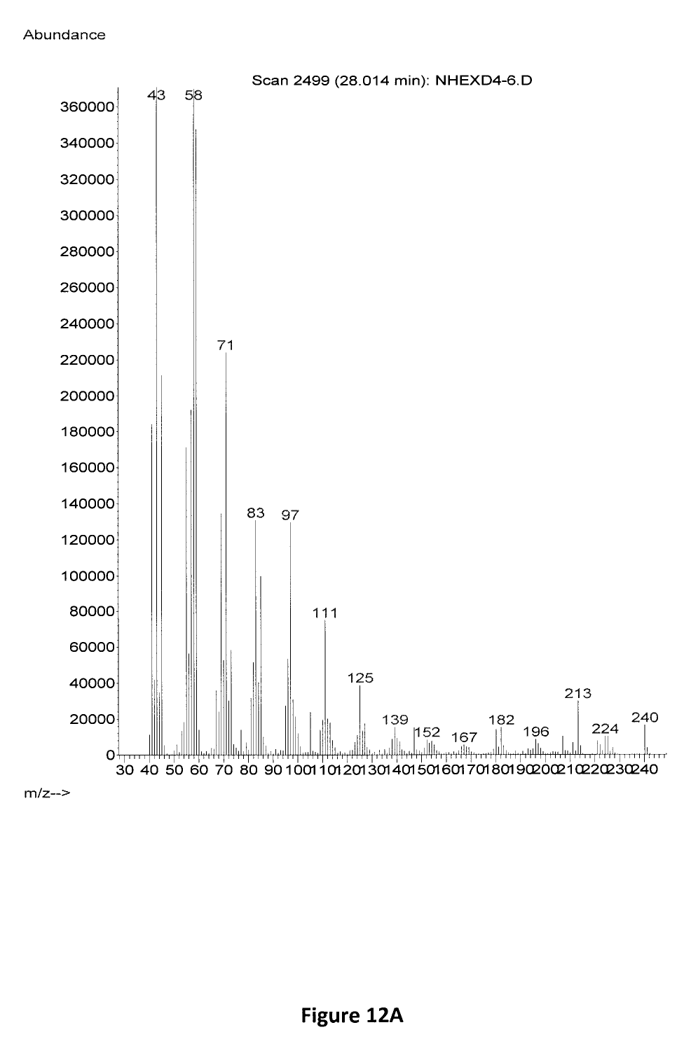

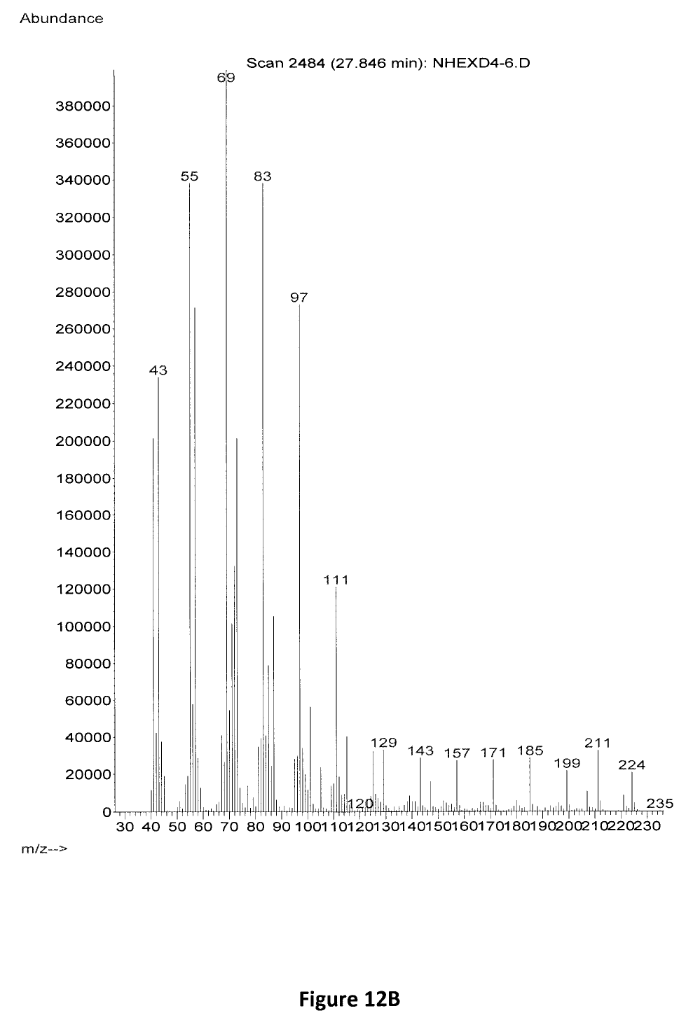

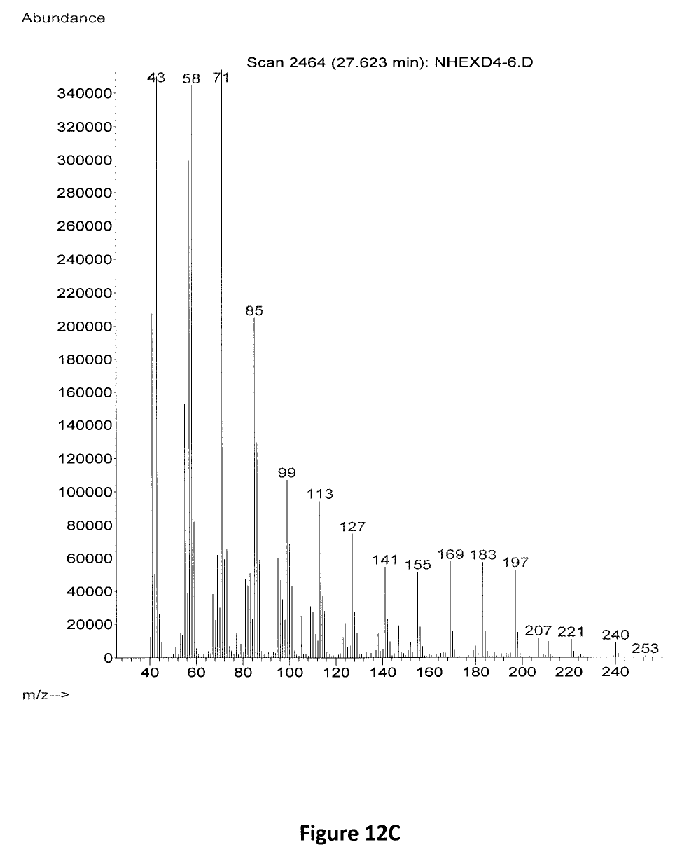

To fully compare the relative catalytic activities of Au/SBA-15 materials with these previous reports, additional study of oxidation n-hexadecane using Au/SBA-15 as catalyst was performed (Table 5). While the Au/SBA-15 catalyzed also the oxidation of n-hexadecane, interestingly, it gave exclusively ketone products, with no alcohol or other oxidized products (Chen et al. (2009) J. Am. Chem. Soc., 131:914-915). Furthermore, the catalytic selectivity of Au/SBA-15 catalyst to particular ketone products was much higher. For instance, Au/SBA-15 catalyst B gave only two or three ketone products, namely 2-hexadecanone and 4-hexadecanone (sometimes with a minor 3-hexadecanone product, depending on the reaction conditions) (Table 5). These products are confirmed by GC and GC-MS which are shown in FIGS. 10 and 11.

Without being bound by theory, this significant catalytic selectivity shown by the instant Au/SBA-15 in n-hexadecane oxidation might be due to three reasons: 1) the size of Au nanoparticle in Au/SBA-15 were higher than that in Chen et al.; 2) the supported Au nanoparticles in Au/SBA-15 do not have strongly bound alkanethiol ligands around them or are ‘naked’; and (3) the difference in the type of oxidants employed in the two cases. Although oxygen, a greener oxidant, was successfully used by Chen et al., it gave a mixture of seven different ketones and six different alcohols (Chen et al. (2009) J. Am. Chem. Soc., 131:914-915). On the other hand, TBHP, which is a less ‘greener’ oxidant, was employed herein, but it gave much more selective products consisting of only two or three different ketones, with no alcohol byproduct.

The products obtained from the catalytic oxidation of n-hexadecane using Au/SBA-15 catalyst were rather similar to those reported for catalytic ozonation of n-hexadecane by activated charcoal or 0.5% Pd-, Ni-, and V-loaded microporous ZSM-5 catalysts (Rajasekhar et al. (2009) Ind. Eng. Chem. Res., 48:9097-9105). In the latter case, only three ketones, that is, 4-hexadecanone, 3-hexadecanone and 2-hexadecanone were reported. In addition, their results showed that 4-hexadecanone was the major product while 3-hexadecanone and 2-hexadecanone were produced in roughly the same, but less significant, amounts. Herein, 3-hexadecanone was sometimes not observed at all, depending on the reaction conditions, while either 4-hexadecanone or 2-hexadecanone was formed as major products (see Table 5).

The recyclability of the Au/SBA-15 catalyst for multiple uses in alkane oxidations was also studied (Table 6). The catalytic selectivity of Au/SBA-15 to produce ketone products remained unchanged or still very high, even after the catalyst was recycled a few times. However, its catalytic activity showed significant reduction, especially after the third cycle. Nevertheless, the amount of Au leached into the solution was very minimal, as characterized by ICP-AES analysis. For instance, after three reaction cycles, the amount of Au in the sample decreased from 196 to 192 μmol Au per gram of Au/SBA-15 (B). In addition, the amount of Au in the reaction mixture was obtained to be only ˜409 ppm (˜3.84 μmol). Thus, the loss of the catalytic activity of the catalyst is probably mainly due to the inactivation or possible pore clogging of the mesoporous channels of the material.

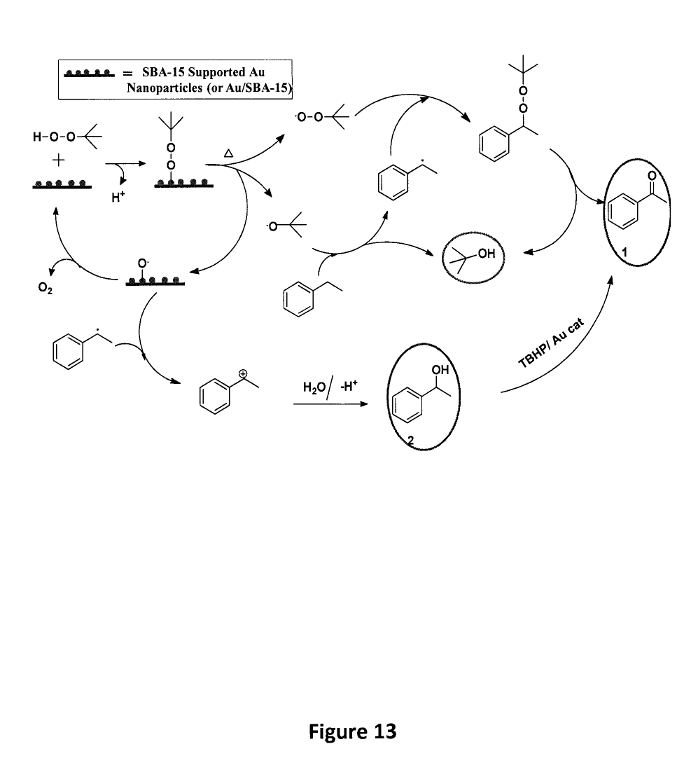

Without being bound by theory, FIG. 13 provides a mechanism for the Au/SBA-15 catalyzed oxidation reaction. The oxidant TBHP is well known to undergo radical chemistry (Barton et al. (1998) New J. Chem., 22:565-568; Barton et al. (1998) New J. Chem., 22:559-563; Barton et al. (1998) Tetrahedron 54:15457-15468; Liu et al. (2010) Chem. Commun., 46:550-552; Li, Y. F. (2007) SYNLETT., 2922-2923; Mendez et al. (2010) Dalton Trans., 39:8457-8463; Mitsudome et al. (2009) Adv. Synth. Catal., 351:1890-1896). In experiments involving addition of a radical scavenger TEMPO in the middle of the reaction, the alkane oxidative reaction in the presence of the Au/SBA-15 catalyst stopped immediately. This suggested that the Au/SBA-15-catalyzed reaction, not surprisingly, goes through radical intermediates. Notably, some TBHP underwent decomposition into t-BuOH in the presence of Au/SBA-15 in the control experiment. Furthermore, catalyst B in the presence of two equivalent of TBHP as oxidant was found to form 79% conversion of ethylbenzene and 93% selectivity to acetophenone product, along with a minor (7%) 1-phenylethanol byproduct (Table 2). On the other hand, the same reaction with only one equivalent of TBHP also produced a very similar selectivity (93%) of acetophenone and 7% of 1-phenylethanol byproduct, despite this reaction gave a lower (51%) conversion of ethylbenzene compared to the reaction with two equivalents of TBHP. The fact that both reactions, at lower and higher TBHP, gave acetophenone (1) and 1-phenylethanol (2) in about similar proportions starting in the early period of the reactions, regardless of the amount of TBHP used, suggests that both 1 and 2 form in parallel via two different mechanisms (instead of the formation of 2 first, followed by its conversion into 1).

Thus, without being bound by theory, the mechanism of Au/SBA-15 catalyzed alkane oxidation probably starts with Au nanoparticle catalyzed decomposition of TBHP (t-BuOOH) into t-BuOO. or t-BuO. radical species. This will be followed by two different TBHP-catalyzed oxidation reactions, producing ketones and secondary alcohols, from the alkene. The t-BuOH is not expected to deactivate the Au nanocatalysts. In fact, alcohols are sometimes used as solvents for Au-catalyzed oxidation reactions of other substances such as alkanes as shown in Table 5 or even oxidation of other alcohols (Mitsudome et al. (2009) Adv. Synth. Catal., 351:1890-1896). Thus, the t-BuOH byproduct from the oxidation reaction would not deactivate the catalyst; however, if it were used as a solvent in larger quantity, it may lower the Au nanoparticles' catalytic activity compared to other solvents, as shown in Table 5, but not when formed as a byproduct.

In conclusion, mesoporous silica-supported nanosized Au particles (Au/SBA-15) have been synthesized. Their use as efficient and selective catalysts for oxidation of ethylbenzene, various other alkyl-substituted benzenes, and different n-alkanes has been demonstrated. The Au/SBA-15 catalysts were shown to oxidize these reactants with TBHP as oxidant and give predominantly the corresponding ketones under the reaction conditions employed. Interestingly, the Au/SBA-15 catalysts generated the ketone products selectively without requiring additives such as carboxylic acids, which are often used for favoring selective oxidation of alkanes into ketone products. The Au/SBA-15 materials were also shown to be versatile selective oxidation catalysts as they successfully catalyzed a series of other alkyl-substituted benzenes such as propylbenzene and diphenylmethane, yielding their corresponding ketone products with high conversion and selectivity. The Au/SBA-15 catalysts also catalyzed n-alkanes including n-hexane and n-hexadecane, resulting in unprecedented higher selectivities to their corresponding ketone products. In addition, the Au/SBA-15 catalyst gave good catalytic activities and very good selectivities in at least three catalytic cycles.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanoparticle comprising a mesoporous silica particle and Au nanoparticles, wherein said Au nanoparticles are contained within the mesopores of said mesoporous silica particle, and wherein the surface of the mesopores of the mesoporous silica particle comprises a reducing agent.

2. The nanoparticle of claim 1, wherein said mesoporous silica is SBA-15.

3. The nanoparticle of claim 1, wherein said reducing agent is a hemiaminal group.

4. The nanoparticle of claim 1, wherein said reducing agent is an imine group.

5. The nanoparticle of claim 1, wherein said Au nanoparticles have a diameter of about 3 nm to about 10 nm.

6. The nanoparticle of claim 1, wherein said mesoporous silica particle comprises capping groups on its external surface.

7. The nanoparticle of claim 6, wherein said capping group is a methyl group.

8. The nanoparticle of claim 6, wherein said capping group is an n-alkyl group.

9. A method of synthesizing the nanoparticle of claim 1 comprising contacting mesoporous silica particles with oxidized Au, wherein the surface of the mesopores of the mesoporous silica particle comprises a reducing agent.

10. The method of claim 9, wherein said reducing agent is a hemiaminal group or an imine group.

11. The method of claim 9, wherein said oxidized gold is Au(III) or Au(I).

12. The method of claim 9, further comprising synthesizing said mesoporous silica particles by a) synthesizing silica particles in the presence of a surfactant, b) grafting the external surface of the silica particles with capping groups, c) removing the surfactant, and d) functionalizing the mesopores with a reducing agent.

13. A method of catalyzing a chemical reaction, said method comprising adding at least one nanoparticle of claim 1 to said chemical reaction.

14. The method of claim 13, wherein said chemical reaction is an oxidation reaction.
